(19) United States
(12) Patent Application Publication
(10) Pub. No.: US 2026/0051391 A1
(43) Pub. Date: Feb. 19, 2026

(54) METHOD FOR ANALYZING A TEXTURE OF A BONE FROM A DIGITIZED IMAGE

(71) Applicant: MEDIMAPS GROUP SA, PLAN-LES-OUATES (CH)

(72) Inventors: Didier HANS, GENTHOD (CH); Lionel BEAUGÉ, SAINT JULIEN EN GENEVOIS (FR); Guillaume GATINEAU, SAINT JULIEN EN GENEVOIS (FR); Franck MICHELET, PESSAC (FR); El Hassen Ahmed LEBRAHIM, CANÉJAN (FR)

(73) Assignee: MEDIMAPS GROUP SA, PLAN-LES-OUATES (CH)

(21) Appl. No.: 18/707,391

(22) PCT Filed: Nov. 2, 2022
(86) PCT No.: PCT/EP2022/080505

§ 371 Date: May 3, 2024

(30) Foreign Application Priority Data
Nov. 3, 2021 (WO) PCT/EP2021/080527

(51) Int. Cl. G16H 30/20; G06T 7/00; G16H 50/20; G16H 50/30

(52) U.S. Cl. CPC G16H 30/20; G06T 7/0012; G16H 50/20; G16H 50/30; G06T 2207/10116; G06T 2207/20081; G06T 2207/20084; G06T 2207/30008

(57) ABSTRACT

A method for analyzing a texture of a bone, including: receiving an input x-ray image showing an input bone, and a bone score analysis of the received input x-ray image by a bone score artificial intelligence implemented by a technical element. The bone score artificial intelligence provides as a result of this bone score analysis: a global score depending at least on a trabecular bone score depending on a texture of the trabecular part of the input bone showed on the received input x-ray image, and/or a trabecular bone score depending on a texture of the trabecular part of the input bone showed on the received input x-ray image. Also, a corresponding device for carrying out the method.

### TECHNICAL FIELD

The present invention relates to a method for analyzing a texture of a bone from a digitized image.

The invention also relates to a device for analyzing a texture of a bone from a digitized image.

The technical field of the invention is typically, but not limited to, the technical field of deep learning in particular to a method for identifying individuals, from opportunistic screening of digital x-ray-based image(s), likely to be diagnosed as osteoporotic with degraded bone quantity and bone microarchitecture as assessed by DXA BMD and TBS or any other equivalent method.

### STATE OF THE ART

In early nineties, the World Health Organization (WHO) defines osteoporosis conceptually as a systemic skeletal disease characterized by low bone mass (decreased quantity) and microarchitectural deterioration of bone tissue (decreased quality) with a consequent increase in bone fragility and susceptibility to fracture (Consensus development conference: diagnosis, prophylaxis, and treatment of osteoporosis. Am J Med—1993-94, 646-650). It has been further defined early 2000 by the NIH—National Institutes of Health—(Osteoporosis prevention, diagnosis, and therapy. Jama. 2001; 285(6):785-95.) as a skeletal disorder characterized by compromised bone strength predisposing to an increased risk of fracture. In essence, in osteoporosis, the deteriorated bone strength leads to the traumatic outcome of fragility fracture.

Bone strength reflects the integration of two main features: bone quantity (i.e. bone density) and bone quality. Bone density is expressed as grams of mineral per area or volume and in any given individual is determined by peak bone mass and amount of bone loss. Bone quality refers to bone architecture, bone resilience, turnover, damage accumulation (e.g., microfractures) and mineralization. Bone architecture is a generic term used for many different entities and can be further refined. At the macroscopic level, bone architecture, known as bone macro-structure (also referred as bone macro-architecture) describes the overall shape and geometry of bone as well as the differentiation into cancellous (also referred to as trabecular) and cortical bone. (Macro- and Microimaging of Bone Architecture, Engelke K et al., in Principles of Bone Biology (Third Edition) Volume II, 2008, Pages 1905-1942. Editors: John Bilezikian Lawrence Raisz T. John Martin-Publisher: Academic Press. Published Date: 29th September 2008).

At any rate, the hallmark of osteoporosis is fragility fracture-defined as a fracture happening due to falls from a standing height in response to mechanical forces that would not normally result in fracture. (Cummings S R et al. Epidemiology and outcomes of osteoporotic fractures. Lancet (London, England). 2002; 359(9319):1761-7. Warriner A H et al., Minor, major, low-trauma, and high-trauma fractures: what are the subsequent fracture risks and how do they vary? Current osteoporosis reports. 2011; 9(3):122-8.) Hip, spine, humerus and forearm are the most common skeletal sites where fragility fractures happen. Those fractures are referred as major osteoporotic fractures.

Once the fracture risk is identified, prevention steps are taken on a certain ‘hierarchical’ level: general lifestyle advices; adequate calcium and/or vitamin D supplementation intake in addition to a healthy lifestyle; and/or pharmacological therapy. Based on the mechanism of function, there are two types of anti-osteoporotic pharmacological therapies: antiresorptive agents (such as bisphosphonates, estrogen agonists/antagonists, estrogens, calcitonin and denosumab) which reduce bone resorption; and anabolic agents (such as teriparatide) which stimulate bone formation. More recently, romosozumab has been approved for its bone forming effects. (Tu K N., et al. Osteoporosis: A Review of Treatment Options. P & T: a peer-reviewed journal for formulary management. 2018; 43(2):92-104.)

More than 9 million fragility fractures happen in the world annually, a number expected to increase given the ageing of the populations (Johnell O., et al. An estimate of the worldwide prevalence and disability associated with osteoporotic fractures. Osteoporosis international: a journal established as result of cooperation between the European Foundation for Osteoporosis and the National Osteoporosis Foundation of the USA. 2006; 17(12):1726-33; John A. Kanis J A, et al. SCOPE 2021: a new scorecard for osteoporosis in Europe. Archives of Osteoporosis (2021) 16:82). It is estimated that after the age of 50 years, one in two women and one in four men will suffer a major osteoporotic fracture in their remaining lifetime. (Kanis J A., et al. Long-term risk of osteoporotic fracture in Malmö. Osteoporosis international: a journal established as result of cooperation between the European Foundation for Osteoporosis and the National Osteoporosis Foundation of the USA. 2000; 11(8):669-74.) In women over 45 years of age, osteoporosis accounts for more days spent in hospital than many other diseases, including diabetes, myocardial infarction, and breast cancer. Moreover, a prior fracture is associated with an 86% increased risk of a subsequent fracture. Fractures are associated with high morbidity and mortality; and are often a precursor of disability, loss of independence, and premature death among the elderly. (Binkley N., et al. Osteoporosis in Crisis: It's Time to Focus on Fracture. Journal of bone and mineral research: the official journal of the American Society for Bone and Mineral Research. 2017; 32(7):1391-4.) For example, in overall, in the EU alone in 2010, the cost of osteoporosis, including pharmacological intervention, was estimated at €37 billion—out of which: 66% represented costs of treating incident fractures, 5% pharmacological prevention and 29% long-term fracture care. (Hernlund E., et al. Osteoporosis in the European Union: medical management, epidemiology, and economic burden. A report prepared in collaboration with the International Osteoporosis Foundation (IOF) and the European Federation of Pharmaceutical Industry Associations (EFPIA). Archives of osteoporosis. 2013; 8(1-2):136.)

Unfortunately, it has been estimated that about 70% of individuals who could be considered at risk of osteoporosis has never been identified nor even referred to bone specialist for appropriate diagnosis by DXA (gold standard). Moreover, many studies are showing that the percentage of patients receiving a registered therapy for osteoporosis, even after sustaining a hip fracture, has declined significantly over the years. In fact, the great majority of individuals at high risk (possibly 80%), who have already had at least one osteoporosis fracture, are neither identified nor treated. (Hernlund, E., et al., Osteoporosis in the European Union: medical management, epidemiology and economic burden. A report prepared in collaboration with the International Osteoporosis Foundation (IOF) and the European Federation of Pharmaceutical Industry Associations (EFPIA). Arch Osteoporos, 2013. 8: p. 136; Solomon D H., et al. Osteoporosis medication use after hip fracture in U.S. patients between 2002 and 2011. J Bone Miner Res. 2014; 29(9): 1929-1937; Kanis J A., et al. SCOPE 2021: a new scorecard for osteoporosis in Europe. Archives of Osteoporosis (2021) 16:82. Bone health and osteoporosis: a report of the Surgeon General.—Rockville, Md.: U.S. Dept. of Health and Human Services, Public Health Service, Office of the Surgeon General; Washington, D.C.: For sale by the Supt. of Docs., U.S. G.P.O., 2004. p. 436).

There are many reasons regarding the under identification of individuals at risk of osteoporosis as well as the decline in the diagnosis and treatment of osteoporosis. Perhaps the biggest problem is a lack of awareness of bone disease among both the public and health care professionals, many of whom do not understand the magnitude of the problem, let alone the ways in which bone disease can be prevented and treated. Even when the patients would have a fragility fracture, there is an underappreciation of the seriousness of all osteoporotic fractures, including asymptomatic vertebral compression fractures, and the failure to ensure that patients admitted to hospital facilities with osteoporotic fractures are directed into an osteoporosis management plan to prevent a second fracture. In fact, to address this later issue and subsequently to avoid a second fragility fracture, there is an international movement to develop multidisciplinary Fracture Liaison Services (FLS). The FLS relies on developing mechanisms and pathways to identify patients admitted to hospitals, emergency rooms, or urgent care clinics with osteoporotic fractures and direct those patients into a well-developed osteoporotic management and treatment plan (Paul D. Miller. Underdiagnoses and Undertreatment of Osteoporosis: The Battle to Be Won. J Clin Endocrinol Metab, March 2016, 101(3):852-859).

There are a number of “red flags” that might signal potential problems with an individual's bone health at different ages before a fragility fracture occurs. While Bone Mineral Density (BMD) testing still serves as the “gold standard” diagnostic test for identifying osteoporosis and fracture risk, population-wide BMD testing is not a cost effective or practical method for assessing the risk of bone disease. While BMD testing has been recommended for some populations (women over age 65), BMD tests are not routinely used for other individuals, the vast majority of whom do not have and are not at risk for bone disease. Widespread BMD testing makes little economic or medical sense. Rather, the evidence supports the assessment of other risk factors first, in order to identify a subset of at-risk individuals who are most likely to benefit from the test (e.g., younger women with multiple risk factors and both men and women who have had fragility fractures or who have diseases that can greatly increase fracture risk). Some of these risk factors may act directly or indirectly to affect BMD levels, but others are independent of bone density (e.g., risk factors for falling). As such many screening strategies based on risk assessment model have been developed. Unfortunately, some problems and limitations have slowed the development and widespread application of risk-factor assessment tools. One important issue relates to limitations of current medical knowledge about risk factors for bone health but more importantly it still requires an active screening behavior which is again related to awareness of both the public and health care professionals (Pal B. Questionnaire survey of advice given to patients with fractures. BMJ 1999 Feb. 20; 318(7182):500-1; Bone health and osteoporosis: a report of the Surgeon General.—Rockville, Md.: U.S. Dept. of Health and Human Services, Public Health Service, Office of the Surgeon General; Washington, D.C.: For sale by the Supt. of Docs., U.S. G.P.O., 2004. p. 436; Kanis J A., et al. SCOPE 2021: a new scorecard for osteoporosis in Europe. Archives of Osteoporosis (2021) 16:82).

To overcome this necessity of pro-activeness and avoid the excess of cost of a systematic screening of the whole population based on DXA for example, one could imagine an automatic opportunistic approach when appropriate. This later one could for example be performed on X-ray based image which are performed on millions of individuals each year for other-than-osteoporosis reasons. Such approach would have to be optimized in term of false positive and should be aimed at increasing the number of individuals at real risk of osteoporosis to be refer to a DXA facility for “gold standard” diagnosis confirmation (both quantity and quality assessment).

The goal of the invention is to present a method or device for analyzing a texture and/or health status of a bone quickly and simply, that can be applied even on an image that would normally not allow to obtain a Bone Mineral Density (BMD) or a Trabecular Bone Score (TBS), i.e. typically that can be applied on other images than Dual-energy X-ray Absorptiometry (DXA) images.

### SUMMARY OF THE INVENTION

An aspect of the invention concerns a (preferably computer implemented) method for analyzing a texture of a bone (preferably from a digitized image, obtained by imaging and chosen in a region comprising a bone structure), comprising:
receiving a digitized input x-ray image showing an input bone,
a bone score analysis of the received input x-ray image by a bone score artificial intelligence implemented by technical means, the bone score artificial intelligence giving as a result of this bone score analysis:
   a global score depending at least on a trabecular bone score (TBS) depending on a texture (or which quantifies the local variations in gray levels from the experimental variogram of the gray levels) of the trabecular part of the input bone showed on the received input x-ray image, and/or
   a trabecular bone score (TBS) depending on a texture (or which quantifies the local variations in gray levels from the experimental variogram of the gray levels) of the trabecular part of the input bone showed on the received input x-ray image and/or a density score depending on a bone mineral density of the input bone showed on the received input x-ray image.

The bone score artificial intelligence can be a neural network.

The method according to the invention can comprise:
constructing a first training set by implementing several times the following steps:
   Obtaining a first type of training image showing a trabecular part of a training bone
   Obtaining an associated second type of training image that is a x-ray based image, showing the same training bone
   Determining, by technical means, from the first type of training image:
      a density score depending on a bone mineral density of the training bone showed on the first type of training image, and
      a trabecular bone score (TBS) depending on a texture (or which quantifies the local variations in gray levels from the experimental variogram of the gray levels) of the trabecular part of the training bone showed on the first type of training image
   Determining, by technical means, from:
      the density score depending on a bone mineral density of the training bone showed on the first type of training image, and
      the trabecular bone score (TBS) depending on a texture (or which quantifies the local variations in gray levels from the experimental variogram of the gray levels) of the trabecular part of the training bone showed on the first type of training image a global score depending on these density score and trabecular bone score
training the bone score artificial intelligence by providing to the bone score artificial intelligence the second type of training image with its associated ground truth comprising the global score determined for the training image of the first type associated with this training image of the second type.

The method according to the invention can comprise:
constructing a second training set by implementing several times the following steps:
   Obtaining a first type of training image showing a trabecular part of a training bone
   Obtaining an associated second type of training image that is a x-ray based image, showing the same training bone
   Determining, by technical means, from the first type of training image:
      a density score depending on a bone mineral density of the training bone showed on the first type of training image, and/or
      a trabecular bone score (TBS) depending on a texture (or which quantifies the local variations in gray levels from the experimental variogram of the gray levels) of the trabecular part of the training bone showed on the first type of training image
training the bone score artificial intelligence by providing to the bone score artificial intelligence the second type of training image with its associated ground truth comprising:
   the density score determined for the training image of the first type associated with this training image of the second type and/or
   the trabecular bone score determined for the training image of the first type associated with this training image of the second type.

The method according to the invention can comprise:
receiving the input x-ray image showing an input bone,
a first analysis of the received input x-ray image by a first artificial intelligence implemented by technical means, the first artificial intelligence giving as a result of the first analysis a global score depending both:
   on a density score depending on a bone mineral density of the input bone showed on the received input x-ray image
   on a trabecular bone score (TBS) depending on a texture (or which quantifies the local variations in gray levels from the experimental variogram of the gray levels) of the trabecular part of the input bone showed on the received input x-ray image
a second analysis of the received input x-ray image by a second artificial intelligence implemented by technical means, the second artificial intelligence giving as a result of the second analysis:
   the density score depending on a bone mineral density of the input bone showed on the received input x-ray image, and/or
   the trabecular bone score (TBS) depending on a texture (or which quantifies the local variations in gray levels from the experimental variogram of the gray levels) of the trabecular part of the input bone showed on the received input x-ray image
a third analysis, by a third artificial intelligence implemented by technical means, the third artificial intelligence having as input the results of the first and second analysis and having as output a result depending on the consistency between the result of the first analysis and the result of the second analysis.

The third artificial intelligence can use as further input at least one parameter among: age of the patient on whom the received input x-ray image was acquired, gender of the patient on whom the received input x-ray image was acquired, morphotype of the patient on whom the received input x-ray image was acquired, machine type with which the received input x-ray image was acquired, and/or acquisition parameter(s) of the machine with which the received input x-ray image was acquired.

The first artificial intelligence can be a neural network and/or the second artificial intelligence can be a neural network.

The first artificial intelligence and the second artificial intelligence and the third artificial intelligence can be three distinct artificial intelligences.

The technical means for implementing the first and second and third artificial intelligences can be the same technical means

The method according to the invention can comprise:
constructing a first training set by implementing several times the following steps:
   Obtaining a first type of training image showing a trabecular part of a training bone
   Obtaining an associated second type of training image that is a x-ray based image, showing the same training bone
   Determining, by technical means, from the first type of training image:
      a density score depending on a bone mineral density of the training bone showed on the first type of training image, and
      a trabecular bone score (TBS) depending on a texture (or which quantifies the local variations in gray levels from the experimental variogram of the gray levels) of the trabecular part of the training bone showed on the first type of training image
   Determining, by technical means, from:
      the density score depending on a bone mineral density of the training bone showed on the first type of training image, and
      the trabecular bone score (TBS) depending on a texture (or which quantifies the local variations in gray levels from the experimental variogram of the gray levels) of the trabecular part of the training bone showed on the first type of training image
   a global score depending on these density score and trabecular bone score
training the first artificial intelligence by providing, to the first artificial intelligence the second type of training image with its associated ground truth comprising the global score determined for the training image of the first type associated with this training image of the second type.

The method according to the invention can comprise:
constructing a second training set by implementing several times the following steps:
   Obtaining a first type of training image showing a trabecular part of a training bone
   Obtaining an associated second type of training image that is a x-ray based image, showing the same training bone
   Determining, by technical means, from the first type of training image:
      a density score depending on a bone mineral density of the training bone showed on the first type of training image, and/or
      a trabecular bone score (TBS) depending on a texture (or which quantifies the local variations in gray levels from the experimental variogram of the gray levels) of the trabecular part of the training bone showed on the first type of training image
training the second artificial intelligence by providing, to the second artificial intelligence the second type of training image with its associated ground truth comprising:
   the density score determined for the training image of the first type associated with this training image of the second type and/or
   the trabecular bone score determined for the training image of the first type associated with this training image of the second type.

The first artificial intelligence and the second artificial intelligence can be trained using a same database of first type of training images and second type of training images.

The method according to the invention can comprise:
constructing a third training set by implementing several times the following steps:
   Obtaining a first type of training image showing a trabecular part of a training bone
   Obtaining a second type of training image that is a x-ray based image, showing the same training bone
   Determining, by technical means, from the first type of training image:
      a density score depending on a bone mineral density of the training bone showed on the first type of training image, and
      a trabecular bone score (TBS) depending on a texture (or which quantifies the local variations in gray levels from the experimental variogram of the gray levels) of the trabecular part of the training bone showed on the first type of training image
   Determining, by technical means, from:
      the density score depending on a bone mineral density of the training bone showed on the first type of training image, and
      the trabecular bone score (TBS) depending on a texture (or which quantifies the local variations in gray levels from the experimental variogram of the gray levels) of the trabecular part of the training bone showed on the first type of training image
   a global score depending on this density score and trabecular bone score
   implementing the first and second analysis by the first and second artificial intelligence on the second type of training image, and
training the third artificial intelligence by learning from a difference between the scores obtained from the first type of training image and the scores obtained from the second type of training image of the same training bone.

The first, second and third artificial intelligences can be trained separately.

The first type of training image and the second type of training image can be acquired on the same training bone and are acquired less than 6 months apart.

The first type of training image can be a dual x-ray absorptiometry (DXA) image, a peripheral quantitative computed tomography ((p)QCT) image and/or High Resolution peripheral quantitative computed tomography (HR-pQCT) image, a computerized tomography (CT) image, or a quantitative ultrasound (QUS) image.

The second type of training image is preferably not a dual x-ray absorptiometry (DXA) image, a peripheral quantitative computed tomography ((p)QCT) image and/or High Resolution peripheral quantitative computed tomography (HR-pQCT) image, a computerized tomography (CT) image, or a quantitative ultrasound (QUS) image.

The received input x-ray image is preferably not a dual x-ray absorptiometry (DXA) image, a peripheral quantitative computed tomography ((p)QCT) image and/or High Resolution peripheral quantitative computed tomography (HR-pQCT) image, a computerized tomography (CT) image, or a quantitative ultrasound (QUS) image.

The received input x-ray image can be a digital x-ray image, having a spatial resolution of less than 1 mm per pixel.

An other aspect of the invention concerns a computer program comprising instructions which, when executed by a computer, implement the steps of the method according to the invention.

An other aspect of the invention concerns a computer program product comprising instructions which, when the program is executed by a computer, cause the computer to carry out the steps of the method according to the invention.

An other aspect of the invention concerns a computer-readable storage medium comprising instructions which, when executed by a computer, cause the computer to carry out the steps of the method according to the invention.

An other aspect of the invention concerns a device for analyzing a texture of a bone (preferably from a digitized image, obtained by imaging and chosen in a region comprising a bone structure), comprising:
means arranged to and/or programmed to and/or configured to receive a digitized input x-ray image showing an input bone,
technical means arranged to and/or programmed to and/or configured to implement a bone score artificial intelligence arranged to and/or programmed to and/or configured to implement a bone score analysis of the received input x-ray image, the bone score artificial intelligence being arranged to and/or programmed to and/or configured to give as a result of this bone score analysis:
   a global score depending at least on a trabecular bone score (TBS) depending on a texture (or which quantifies the local variations in gray levels from the experimental variogram of the gray levels) of the trabecular part of the input bone showed on the received input x-ray image, and/or
   a trabecular bone score (TBS) depending on a texture (or which quantifies the local variations in gray levels from the experimental variogram of the gray levels) of the trabecular part of the input bone showed on the received input x-ray image and/or a density score depending on a bone mineral density of the input bone showed on the received input x-ray image.

The bone score artificial intelligence can be a neural network.

The device according to the invention can comprise:
means arranged to and/or programmed to and/or configured to construct a first training set by implementing several times the following steps:
   Obtaining (by technical means for obtaining) a first type of training image showing a trabecular part of a training bone
   Obtaining (by technical means for obtaining) an associated second type of training image that is a x-ray based image, showing the same training bone
   Determining, by technical means for determining, from the first type of training image:
      a density score depending on a bone mineral density of the training bone showed on the first type of training image, and
      a trabecular bone score (TBS) depending on a texture (or which quantifies the local variations in gray levels from the experimental variogram of the gray levels) of the trabecular part of the training bone showed on the first type of training image
   Determining, by technical means for determining, from:
      the density score depending on a bone mineral density of the training bone showed on the first type of training image, and
      the trabecular bone score (TBS) depending on a texture (or which quantifies the local variations in gray levels from the experimental variogram of the gray levels) of the trabecular part of the training bone showed on the first type of training image
   a global score depending on these density score and trabecular bone score
means arranged to and/or programmed to and/or configured to train the bone score artificial intelligence by providing to the bone score artificial intelligence the second type of training image with its associated ground truth comprising the global score determined for the training image of the first type associated with this training image of the second type.

The device according to the invention can comprise:
means arranged to and/or programmed to and/or configured to construct a second training set by implementing several times the following steps:
   Obtaining (by technical means for obtaining) a first type of training image showing a trabecular part of a training bone
   Obtaining (by technical means for obtaining) an associated second type of training image that is a x-ray based image, showing the same training bone
   Determining, by technical means for determining, from the first type of training image:
      a density score depending on a bone mineral density of the training bone showed on the first type of training image, and/or
      a trabecular bone score (TBS) depending on a texture (or which quantifies the local variations in gray levels from the experimental variogram of the gray levels) of the trabecular part of the training bone showed on the first type of training image
means arranged to and/or programmed to and/or configured to train the bone score artificial intelligence by providing to the artificial intelligence the second type of training image with its associated ground truth comprising:
   the density score determined for the training image of the first type associated with this training image of the second type and/or
   the trabecular bone score determined for the training image of the first type associated with this training image of the second type.

The device according to the invention can comprise:
means arranged to and/or programmed to and/or configured to receive the input x-ray image showing an input bone,
technical means arranged to and/or programmed to and/or configured to implement a first artificial intelligence arranged to and/or programmed to and/or configured to implement a first analysis of the received input x-ray image, the first artificial intelligence being arranged to and/or programmed to and/or configured to give as a result of the first analysis a global score depending both:
   on a density score depending on a bone mineral density of the input bone showed on the received input x-ray image
   on a trabecular bone score (TBS) depending on a texture (or which quantifies the local variations in gray levels from the experimental variogram of the gray levels) of the trabecular part of the input bone showed on the received input x-ray image
technical means arranged to and/or programmed to and/or configured to implement a second artificial intelligence arranged to and/or programmed to and/or configured to implement a second analysis of the received input x-ray image, the second artificial intelligence being arranged to and/or programmed to and/or configured to give as a result of the second analysis:
   the density score depending on a bone mineral density of the input bone showed on the received input x-ray image, and/or
   the trabecular bone score (TBS) depending on a texture (or which quantifies the local variations in gray levels from the experimental variogram of the gray levels) of the trabecular part of the input bone showed on the received input x-ray image
technical means arranged to and/or programmed to and/or configured to implement a third artificial intelligence arranged to and/or programmed to and/or configured to implement a third analysis, the third artificial intelligence being arranged to and/or programmed to and/or configured to have as input the results of the first and second analysis and to have as output a result depending on the consistency between the result of the first analysis and the result of the second analysis.

Third artificial intelligence can be arranged to and/or programmed to and/or configured to use as further input at least one parameter among: age of the patient on whom the received input x-ray image was acquired, gender of the patient on whom the received input x-ray image was acquired, morphotype of the patient on whom the received input x-ray image was acquired, machine type with which the received input x-ray image was acquired, and/or acquisition parameter(s) of the machine with which the received input x-ray image was acquired.

The first artificial intelligence can be a neural network and/or the second artificial intelligence can be a neural network.

The first artificial intelligence and the second artificial intelligence and the third artificial intelligence can be three distinct artificial intelligences.

The technical means for implementing the first and second and third artificial intelligences can be the same technical means

The device according to the invention can comprise:
means arranged to and/or programmed to and/or configured to construct a first training set by implementing several times the following steps:
   Obtaining (by technical means for obtaining) a first type of training image showing a trabecular part of a training bone
   Obtaining (by technical means for obtaining) an associated second type of training image that is a x-ray based image, showing the same training bone
   Determining, by technical means for determining, from the first type of training image:
      a density score depending on a bone mineral density of the training bone showed on the first type of training image, and
      a trabecular bone score (TBS) depending on a texture (or which quantifies the local variations in gray levels from the experimental variogram of the gray levels) of the trabecular part of the training bone showed on the first type of training image
   Determining, by technical means for determining, from:
      the density score depending on a bone mineral density of the training bone showed on the first type of training image, and
      the trabecular bone score (TBS) depending on a texture (or which quantifies the local variations in gray levels from the experimental variogram of the gray levels) of the trabecular part of the training bone showed on the first type of training image
   a global score depending on these density score and trabecular bone score
means arranged to and/or programmed to and/or configured to train the first artificial intelligence by providing, to the first artificial intelligence the second type of training image with its associated ground truth comprising the global score determined for the training image of the first type associated with this training image of the second type.

The device according to the invention can comprise:
means arranged to and/or programmed to and/or configured to construct a second training set by implementing several times the following steps:
   Obtaining (by technical means for obtaining) a first type of training image showing a trabecular part of a training bone
   Obtaining (by technical means for obtaining) an associated second type of training image that is a x-ray based image, showing the same training bone
   Determining, by technical means for determining, from the first type of training image:
      a density score depending on a bone mineral density of the training bone showed on the first type of training image, and/or
      a trabecular bone score (TBS) depending on a texture (or which quantifies the local variations in gray levels from the experimental variogram of the gray levels) of the trabecular part of the training bone showed on the first type of training image
means arranged to and/or programmed to and/or configured to train the second artificial intelligence by providing, to the second artificial intelligence the second type of training image with its associated ground truth comprising:
   the density score determined for the training image of the first type associated with this training image of the second type and/or
   the trabecular bone score determined for the training image of the first type associated with this training image of the second type.

The first artificial intelligence and the second artificial intelligence can be arranged to and/or programmed to and/or configured to be trained by using a same database of first type of training images and second type of training images.

The device according to the invention can comprise:
means arranged to and/or programmed to and/or configured to construct a third training set by implementing several times the following steps:
   Obtaining (by technical means for obtaining) a first type of training image showing a trabecular part of a training bone
   Obtaining (by technical means for obtaining) a second type of training image that is a x-ray based image, showing the same training bone
   Determining, by technical means for determining, from the first type of training image:
      a density score depending on a bone mineral density of the training bone showed on the first type of training image, and
      a trabecular bone score (TBS) depending on a texture (or which quantifies the local variations in gray levels from the experimental variogram of the gray levels) of the trabecular part of the training bone showed on the first type of training image
   Determining, by technical means for determining, from:
      the density score depending on a bone mineral density of the training bone showed on the first type of training image, and
      the trabecular bone score (TBS) depending on a texture (or which quantifies the local variations in gray levels from the experimental variogram of the gray levels) of the trabecular part of the training bone showed on the first type of training image
   a global score depending on this density score and trabecular bone score
   implementing the first and second analysis by the first and second artificial intelligence on the second type of training image, and
means arranged to and/or programmed to and/or configured to train the third artificial intelligence by learning from a difference between the scores obtained from the first type of training image and the scores obtained from the second type of training image of the same training bone.

The first, second and third artificial intelligences can be arranged to and/or programmed to and/or configured to be trained separately.

The means arranged to and/or programmed to and/or configured to train the first artificial intelligence and the means arranged to and/or programmed to and/or configured to train the second artificial intelligence can be arranged together to and/or programmed to and/or configured together to check that the first type of training image and the second type of training image have been acquired on the same training bone and have been acquired less than 6 months apart.

The first type of training image can be a dual x-ray absorptiometry (DXA) image, a peripheral quantitative computed tomography ((p)QCT) image and/or High Resolution peripheral quantitative computed tomography (HR-pQCT) image, a computerized tomography (CT) image, or a quantitative ultrasound (QUS) image.

The second type of training image is preferably not a dual x-ray absorptiometry (DXA) image, a peripheral quantitative computed tomography ((p)QCT) image and/or High Resolution peripheral quantitative computed tomography (HR-pQCT) image, a computerized tomography (CT) image, or a quantitative ultrasound (QUS) image.

The received input x-ray image is preferably not a dual x-ray absorptiometry (DXA) image, a peripheral quantitative computed tomography ((p)QCT) image and/or High Resolution peripheral quantitative computed tomography (HR-pQCT) image, a computerized tomography (CT) image, or a quantitative ultrasound (QUS) image.

The received input x-ray image can be a digital x-ray image, having a spatial resolution of less than 1 mm per pixel.

### DETAILED DESCRIPTION OF THE FIGURES AND OF REALIZATION MODES OF THE INVENTION

Other advantages and characteristics of the invention will appear upon examination of the detailed description of embodiments which are in no way limitative, and of the appended drawings in which:

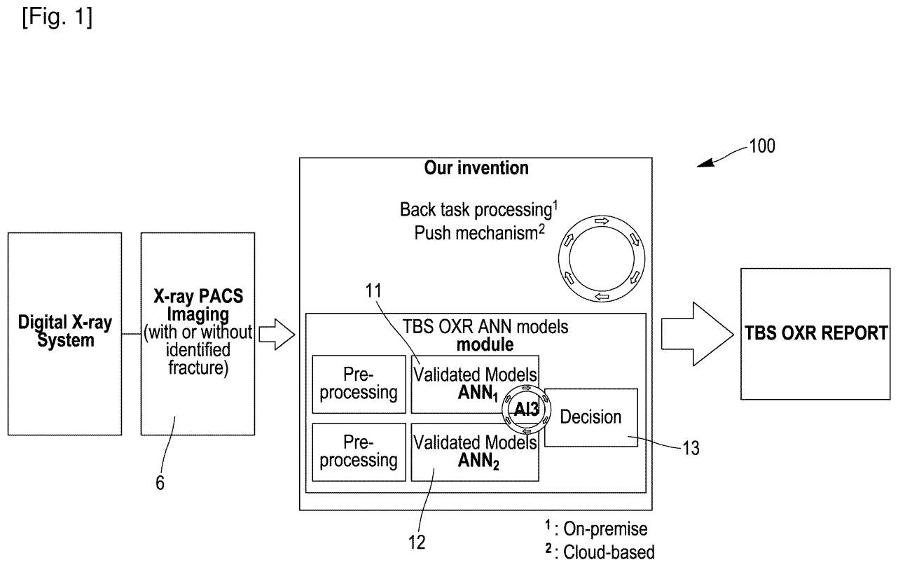

FIG. 1 illustrates some steps of a first embodiment (best realization mode) of a method 100 according to the invention, in an industrialization phase, i.e. the Artificial Neural Network (ANN) model's module within final product of a first embodiment (best realization mode) of a device according to the invention implementing the first embodiment of a method 100 according to the invention,

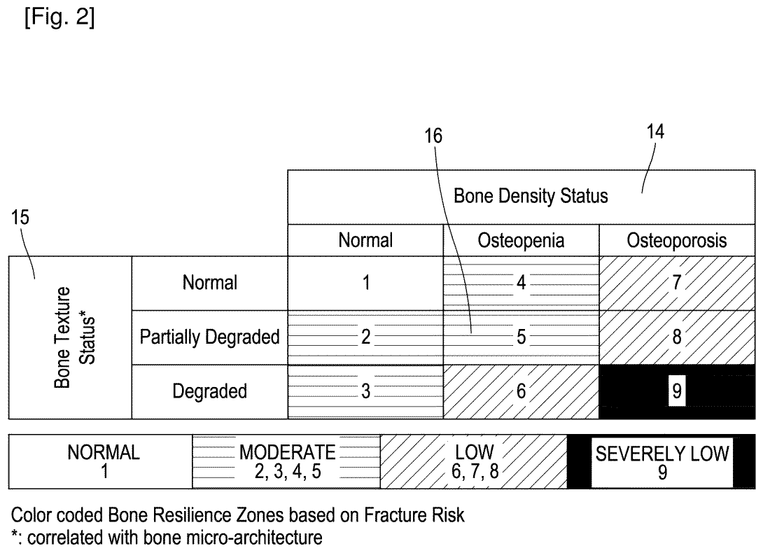

FIG. 2 illustrates the principle of the global score 16 in this embodiment of a method 100 according to the invention, in a table of Bone Health (and associated risk) Classification using Bone Density×Bone Texture (e.g. minimum BMD T-score×TBS), further indicating in its lower part the Bone Health categories based on Fracture Risk,

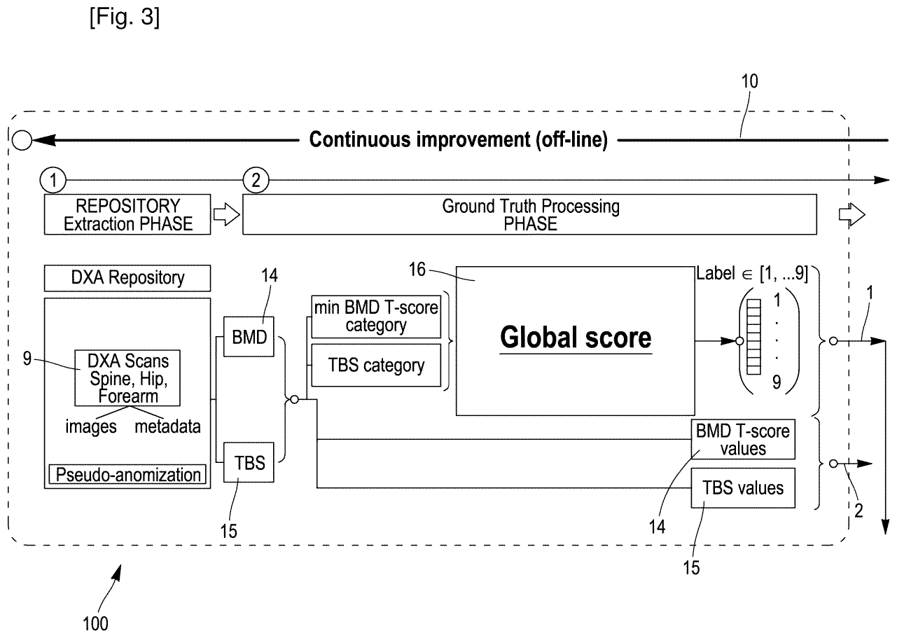

FIG. 3 illustrates some steps of the first embodiment of a method 100 according to the invention, in a repository extraction phase and in a ground truth processing phase,

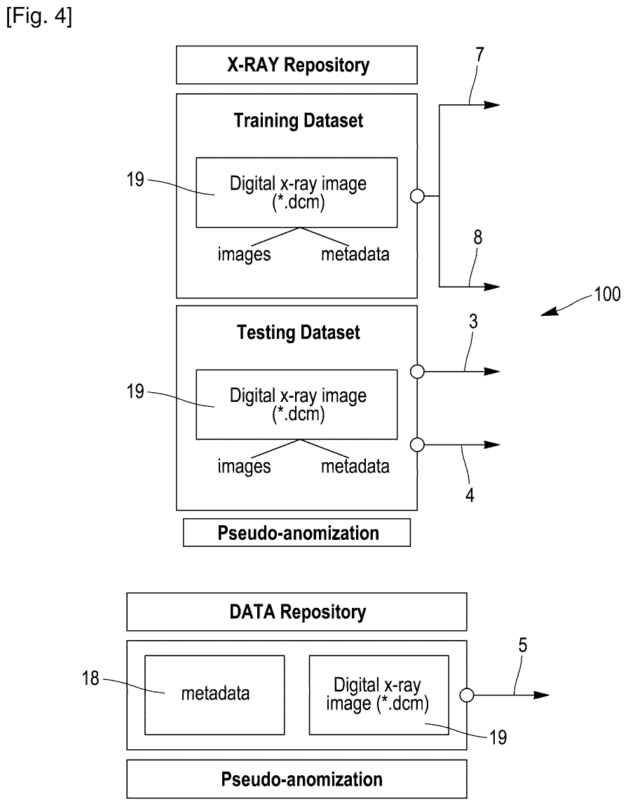

FIG. 4 illustrates some steps of the first embodiment of a method 100 according to the invention, in the repository extraction phase,

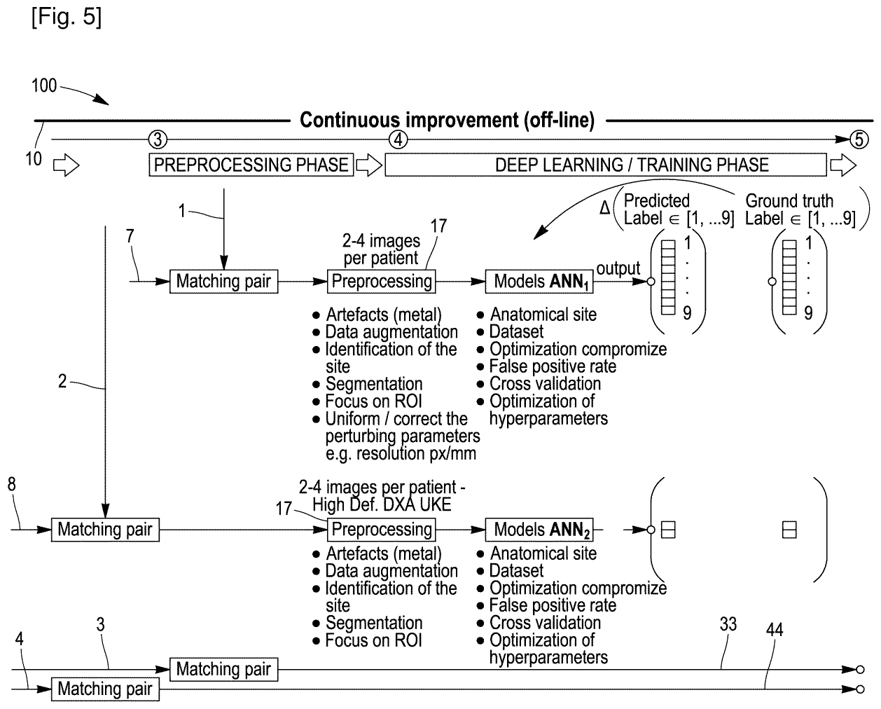

FIG. 5 illustrates some steps of the first embodiment of a method 100 according to the invention, in a preprocessing phase and in a deep learning or training phase,

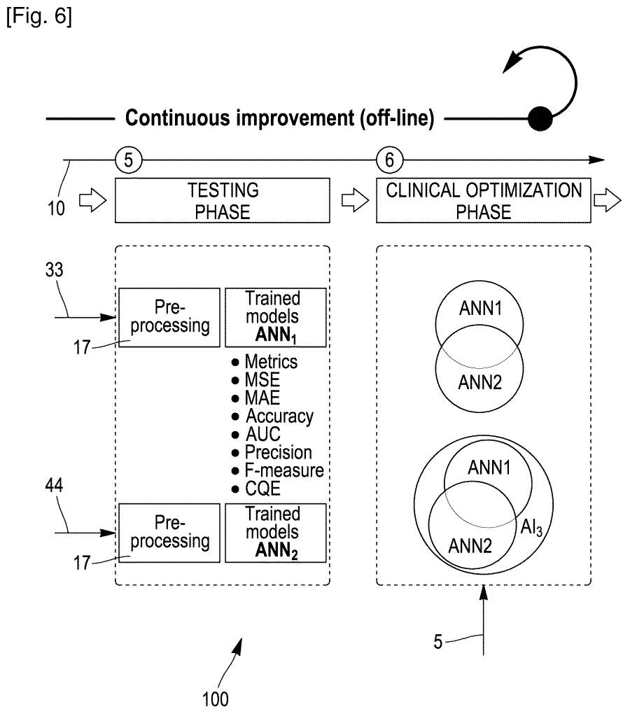

FIG. 6 illustrates some steps of the first embodiment of a method 100 according to the invention, in a testing phase and in a clinical optimization phase,

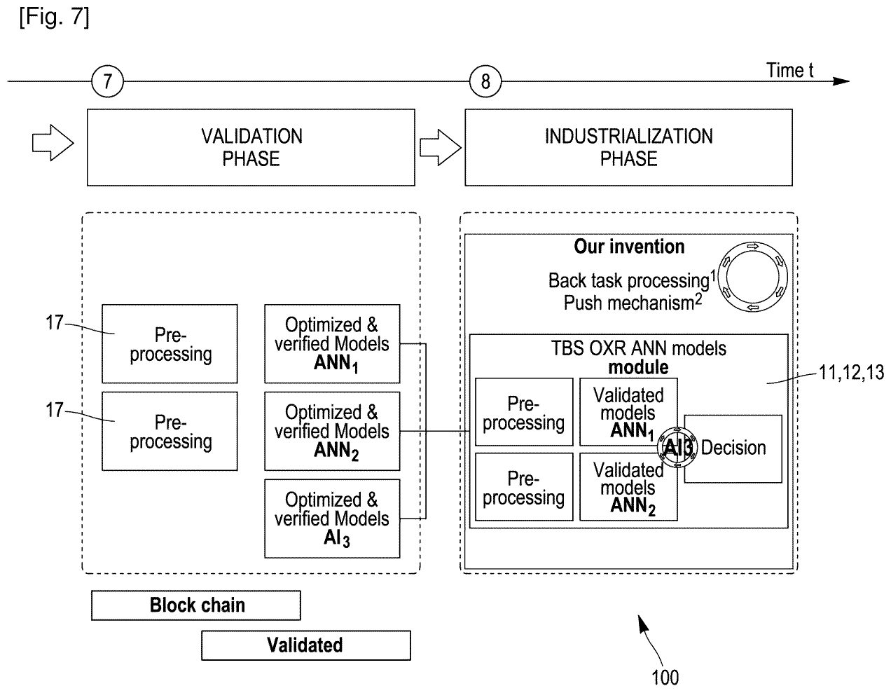

FIG. 7 illustrates some steps of the first embodiment of a method 100 according to the invention, in a validation phase and in the industrialization phase, and

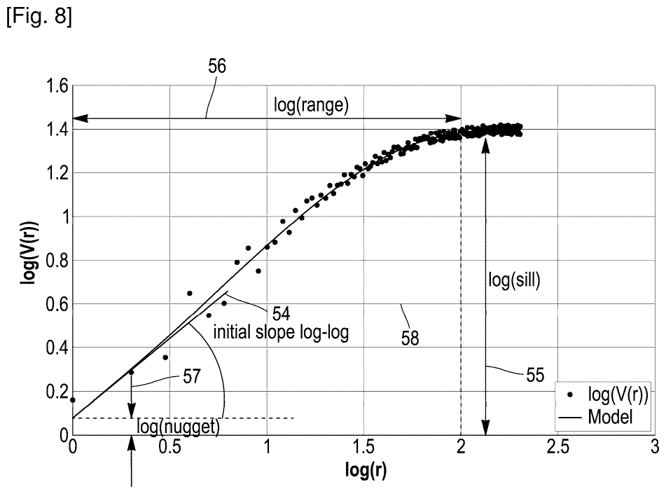

FIG. 8 illustrates an example of variogram V on log-log scale used in the training steps of method 100, with the parameters a, b, c, d and e.

These embodiments being in no way limitative, we can consider variants of the invention including only a selection of characteristics or steps subsequently described or illustrated, isolated from other described or illustrated characteristics or steps (even if this selection is taken from a sentence containing these other characteristics or steps), if this selection of characteristics or steps is sufficient to give a technical advantage or to distinguish the invention over the state of the art. This selection may include at least one characteristic, preferably a functional characteristic without structural details, or with only a part of the structural details if that part is sufficient to give a technical advantage or to distinguish the invention over the state of the art.

We are now going to describe a, in reference to FIGS. 1 to 8, a first embodiment of a computer implemented method 100 according to the invention.

FIGS. 1 to 7 illustrate different parts of this method 100.

These figures are linked with each other.

For example:
link 1 of FIG. 3 corresponds to link 1 of FIG. 5,
link 2 of FIG. 3 corresponds to link 2 of FIG. 5,
link 3 of FIG. 4 corresponds to link 3 of FIG. 5,
link 4 of FIG. 4 corresponds to link 4 of FIG. 5,
link 5 of FIG. 4 corresponds to link 5 of FIG. 6,
link 7 of FIG. 4 corresponds to link 7 of FIG. 5,
link 8 of FIG. 4 corresponds to link 8 of FIG. 5,
link 10 of FIG. 3 corresponds to link 10 of FIGS. 5 and 6,
link 33 of FIG. 5 corresponds to link 33 of FIG. 6,
link 44 of FIG. 5 corresponds to link 44 of FIG. 6.

Method 100 aims at helping the radiologist identify individuals at a potential high risk of osteoporosis as defined by both a low Bone Mineral Density (BMD) and a degraded bone microarchitecture. Those individuals may be referred to bone disease expert center or DXA center for diagnosis confirmation and/or appropriate disease management.

There are two types of bone tissue in the human skeleton, namely cortical and trabecular. The two types of bone differ macroscopically and microscopically, but are identical in their chemical composition. Trabecular bone, also called cancellous bone or spongy bone is a highly porous bone (typically 75-95% porosity) enclosing numerous large spaces that give a honeycombed or spongy like trabecular network with large remodeling area and high turnover rate. More precisely, the bone matrix is organized into a three-dimensional latticework of interconnected rods and plates called trabeculae which surround pores that are filled with bone marrow, fat and blood vessels. This trabecular bone is usually arranged along lines of mechanical stress. Cancellous bone is usually surrounded by a shell of compact (also name cortical) bone, which provides greater strength and rigidity. Cancellous bone makes up about 20 percent of the human skeleton bone mass but about 80% of the bone remodeling activity, providing structural support and flexibility and making this bone the most appropriate target for treatment influencing bone metabolism but also the first bone to be impacted by metabolic disorders such as primary and secondary osteoporosis.

Cancellous bone, otherwise called trabecular bone, is thus a part of bones well known by the one skilled is the art, and is predominant in short bones, such as the vertebrae. It is the main constituent of the body of the vertebrae, the bones of the wrist and the center of the long bones. It is friable, made up of bone lamellae or bone trabeculae arranged in a non-concentric manner around cavities or areolas, filled with red bone marrow. It is therefore:
made up of lamellae or bony trabeculae giving the appearance of a sponge;
less resistant than cortical bone, but offers three-dimensional resistance to the stresses to which it is subjected;
mainly present in the central part of the bones;
very vascularized and in perpetual remodeling in order to adapt the bone structure to the frequency and intensity of shocks;
more prone to fracture by crushing under violent pressure and more affected by osteoporosis.

Method 100 analyses opportunistically digital x-ray images acquired during routine clinical practice from “Picture Archiving and Communication System” (PACS) or cloud-based systems. Those radiographic images are primarily acquired for other-than-osteoporosis reasons. The individuals scanned during this routine practice would usually not be diagnosed for osteoporosis.

Method 100 is a global ensemble model which uses a combination of multiple Artificial Neural Networks (ANN) ANN₁ and ANN₂ to perform the analysis of digital x-ray images.

These ANN are trained using the Bone Mineral Density (BMD) and Trabecular Bone Score (TBS, a texture parameter correlated to bone microarchitecture-cf. patent reference FR_2848694), assessed by Dual-energy X-ray Absorptiometry (DXA), as a ground truth. The combination of BMD and TBS currently constitutes the gold standard for osteoporosis diagnosis.

From the combined outputs of the multiple ANN, a final risk assessment is provided.

Using this method, method 100 performs an opportunistic screening as a systematic background task via the PACS system or as an active push via a cloud-based platform, to identify individuals with high osteoporosis risk.

The global approach of this method 100 is thus an opportunistic screening of the patients using bone related x-ray images from the PACS in a background task manner (or as an active push via a cloud-based platform) to identify individuals most likely to be either at high risk or at a very low risk of osteoporosis as defined from DXA by bone mineral density (BMD) and trabecular bone score (TBS).

For those who are identified at high risk, an optional comprehensive automatic report can be generated, suggesting referral to bone expert center or DXA center for diagnostic confirmation (BMD+TBS).

The approach of method 100 is based on a combination of supervised deep learning models. The Artificial Intelligence (AI) models ANN₁ and ANN₂ of method 100 are trained on ground truth DXA (but not limited) data (BMD+TBS) to process digital x-ray-based images.

This method 100 is optimized for clinical outcome (low rate of false positive, etc.), low processing time, and is seamlessly integrated into the radiological workflow.

Method 100 comprises, in the following order, the following phases:
Repository extraction phase, illustrated in FIGS. 3 (left part) and 4,
Ground truth processing phase, illustrated in FIG. 3 (right part),
Preprocessing phase, illustrated in FIG. 5 (left part),
Deep learning or training phase, illustrated in FIG. 5 (right part),
Testing phase, illustrated in FIG. 6 (left part),
Clinical optimization phase, illustrated in FIG. 6 (right part),
Validation phase, illustrated in FIG. 7 (left part),
Industrialization phase, illustrated in FIG. 7 (right part).

As illustrated in FIG. 1, method 100 comprises (in its final industrialization phase of FIG. 1 and right part of FIG. 7) acquiring and receiving a digitized input x-ray image 6 showing an input bone.

The received input x-ray image 6 is preferably not a dual x-ray absorptiometry (DXA) image, a peripheral quantitative computed tomography ((p)QCT) image and/or High Resolution peripheral quantitative computed tomography (HR-pQCT) image, a computerized tomography (CT) image, or a quantitative ultrasound (QUS) image.

In this embodiment, each image 6, 9 or 19 is a digitized image.

The received input x-ray image 6 is preferably a digital x-ray image, having a spatial resolution per pixel of less than 1 mm.

Method 100 then comprises a first analysis 11 of the received input x-ray image 6 by a first artificial intelligence ANN₁ implemented by technical means (typically comprising or consisting of at least one computer, one central processing or computing unit, one analogue electronic circuit (preferably dedicated), one digital electronic circuit (preferably dedicated) and/or one microprocessor (preferably dedicated) and/or by software means), the first artificial intelligence giving as a result of the first analysis a global score 16, illustrated in FIG. 2, depending both:
on a density score depending on (or consisting of) a bone mineral density of the input bone showed on the received input x-ray image
on a trabecular bone score (TBS) depending on a texture of the trabecular part of the input bone showed on the received input x-ray image

Global score 16 is preferably depending only on this density score (preferably consisting of the bone mineral density) and a trabecular bone score (TBS), and is thus preferably a combination of this density score (preferably consisting of the bone mineral density) and a trabecular bone score (TBS).

Nevertheless, ANN₁ does not determine or calculate the density score or TBS from image 6, but directly determine the global score 16.

The first artificial intelligence is a neural network.

The global score 16 is a Multi Class Classifier.

The global score 16 has a finite number of possible values. It only has 9 possible values, illustrated in FIG. 2. Each of these values is a positive integer. Each of these values is a positive integer, preferably among 1, 2, 3, 4, 5, 5, 7, 8 and 9.

In all this description of FIGS. 1 to 8, the density score can be:
Equal to the bone mineral density
determined according to the value of BMD, for example:
   if BMD T-score≤−2.5 then density score=“Osteoporosis” or “osteoporotic”
   if BMD T-score in]−2.5, −1 [ then density score=“Osteopenia” or “osteopenic”
   if BMD T-score≥−1 then density score=“Normal”;
bone mineral density being determined from image 9 according to classical prior art techniques. Bone Mineral Density (BMD) is typically determined from the absorption of each beam by bone. Dual-energy X-ray absorptiometry is the most widely used and most thoroughly studied bone density measurement technology.

Usually, bone mineral density is retrieved directly from the Dual X-ray Absorptiometry (DXA) image, and can typically be a metadata of the Dual X-ray Absorptiometry (DXA) image itself.

The trabecular bone score (TBS) is a textural parameter which quantifies the local variations in gray levels and is derived from the evaluation of the experimental variogram of the gray levels of a digitized image, this digitized image being typically a Dual X-ray Absorptiometry (DXA) image but can also be other digital X-ray image or many other X-ray image modalities.

More details or example of TBS can be found in the article “Trabecular bone score (TBS) as a new complementary approach for osteoporosis evaluation in clinical practice. A consensus report of a European Society for Clinical and Economic Aspects of Osteoporosis and Osteoarthritis (ESCEO) Working Group” by N. C. Harvey & al. (https://www.ncbi.nlm.nih.gov/pmc/articles/PMC4538791/)

Nevertheless, in this particular case, the global score 16 is determined by the first artificial intelligence without any calculation of a TBS and without any calculation or determination of an experimental variogram of the gray levels of the received input x-ray image 6.

Method 100 then comprises a second analysis 12 of the received input x-ray image 6 by a second artificial intelligence ANN₂ implemented by technical means (typically comprising or consisting of at least one computer, one central processing or computing unit, one analogue electronic circuit (preferably dedicated), one digital electronic circuit (preferably dedicated) and/or one microprocessor (preferably dedicated) and/or by software means), the second artificial intelligence giving as a result of the second analysis:
the density score depending on (or consisting of) a bone mineral density of the input bone showed on the received input x-ray image 6, and/or
the trabecular bone score (TBS) depending on a texture of the trabecular part of the input bone showed on the received input x-ray image 6

The second artificial intelligence is a neural network.

The second artificial intelligence is a regression model that gives or infers continuous values.

The TBS is determined by the second artificial intelligence without any calculation or determination of an experimental variogram of the gray levels of the received input x-ray image 6.

Method 100 then comprises a third analysis 13, by a third artificial intelligence AI₃ implemented by technical means (typically comprising or consisting of at least one computer, one central processing or computing unit, one analogue electronic circuit (preferably dedicated), one digital electronic circuit (preferably dedicated) and/or one microprocessor (preferably dedicated) and/or by software means), the third artificial intelligence having as input the results of the first and second analysis and having as output a result depending on the consistency between the result of the first analysis and the result of the second analysis; typically AI₃ outputs depend on consistency between the results of the first analysis of ANN₁ and the results of the second analysis of ANN₂, with the use of weighted information from patient-related meta-data 18 (such as soft tissue thickness, age, BMI, etc.):
Case #1:
   ANN1=6, 7, 8 or 9 (glob. Score)
   ANN2=bone density status osteopenic or osteoporotic, texture status partially degraded or degraded
then result of AI3=flag
Case #2:
   ANN1=6, 7, 8 or 9 (glob. Score)
   ANN2=bone density status normal, texture status partially degraded or degraded
then result of AI3=weighted response to assess if flag or not, depending on metadata 18
Case #3:
   ANN1=6, 7, 8 or 9 (glob. Score)
   ANN2=bone density status osteopenic or osteoporotic, texture status normal
then result of AI3=weighted response to assess if flag or not, depending on metadata 18
Case #4:
   ANN1=1, 2, 3, 4 or 5 (glob. Score)
   ANN2=bone density status normal, texture status normal then result of AI3=No flag
Case #5
   ANN1=1, 2, 3, 4 or 5 (glob. Score)
   ANN2=bone density status osteopenic or osteoporotic, texture status normal
then result of AI3=weighted response to assess if flag or not, depending on metadata 18
Case #6
   ANN1=1, 2, 3, 4 or 5 (glob. Score)
   ANN2=bone density status, texture status partially degraded or degraded then result of AI3=weighted response to assess if flag or not, depending on metadata 18

The third artificial intelligence uses as further input metadata 18 comprising at least one parameter among: age of the patient on whom the received input x-ray image was acquired, gender of the patient on whom the received input x-ray image was acquired, morphotype of the patient on whom the received input x-ray image was acquired, machine type with which the received input x-ray image was acquired, and/or acquisition parameter(s) of the machine with which the received input x-ray image was acquired.

The third artificial intelligence can be a neural network but is preferably a binary classifier such as decision tree, random forest or support vector machine.

Thus, as a final step, the output of the two previously described approaches ANN₁ and ANN₂ are entered with additional selected meta-data 18 (e.g. age, gender, morphotype, machine type, acquisition parameters) as input variables in AI₃ which is typically a decision tree-like learning model. The classification tree will provide as an output the best combination for final classification optimization (likelihood to be selected as osteoporotic with degraded bone microarchitecture as assessed by DXA BMD and TBS). Some specific techniques, also called ensemble methods will be used such as, but not limited to, bagged decision tree to consider the possibility of multi-image set for a given time point and given individual.

The first artificial intelligence and the second artificial intelligence and the third artificial intelligence are three distinct artificial intelligences or three distinct artificial intelligence architectures.

The technical means for implementing the first and second and third artificial intelligences are preferably the same technical means, i.e preferably but not restricted to, integrated into embedded modules, within the same at least one computer, one central processing or computing unit, one analogue electronic circuit (preferably dedicated), one digital electronic circuit (preferably dedicated) and/or one microprocessor (preferably dedicated) and/or by software means.

Those artificial intelligences ANN₁, ANN₂, AI₃ are previously trained.

Method 100 thus comprises, before the analysis steps 11, 12 and 13 of the industrialization phase, the following steps:
constructing a first training set by implementing several times the following steps:
   Obtaining a first type of training image 9 (of the first training set) showing a trabecular part of a training bone (during the repository extraction phase)
   Obtaining an associated second type 19 of training image (of the first training set) that is a x-ray based image, showing the same training bone, but not necessary its trabecular part (during the repository extraction phase)
   Determining (during the ground truth processing phase), by technical means (typically comprising or consisting of at least one computer, one central processing or computing unit, one analogue electronic circuit (preferably dedicated), one digital electronic circuit (preferably dedicated) and/or one microprocessor (preferably dedicated) and/or by software means), from the first type of training image 9 of the first training set (but without implementing any artificial intelligence on image 9):
      a density score 14 depending on or equal to a bone mineral density of the training bone showed on the first type of training image of the first training set; typically, the bone mineral density is determined from the first type of training image directly as a metadata of the DXA the first type of training image; and
      a trabecular bone score (TBS) 15 depending on a texture of the trabecular part of the training bone showed on the first type of training image of the first training set; typically, this TBS 15 is determined by calculating or determining an experimental variogram of the gray levels of the first type of training image (patent reference EP1576526_A1)
   Determining (during the ground truth processing phase), by technical means (typically comprising or consisting of at least one computer, one central processing or computing unit, one analogue electronic circuit (preferably dedicated), one digital electronic circuit (preferably dedicated) and/or one microprocessor (preferably dedicated) and/or by software means), from:
      the density score 14 depending on or equal to a bone mineral density of the training bone showed on the first type of training image 9 of the first training set, and
      the trabecular bone score (TBS) 15 depending on a texture of the trabecular part of the training bone showed on the first type of training image 9 of the first training set
   (but without implementing any artificial intelligence on image 9, and/or on previously determined density score 14 and/or on previously determined TBS 15) a global score 16 depending on (preferably only on) these density score and trabecular bone score.

For example:
the density score or status can be determined according to the value of Bone Mineral Density (BMD), for example:
   if BMD T-score≤−2.5 then density score=“Osteoporosis” or “osteoporotic”
   if BMD T-score in]−2.5, −1 [ then density score=“Osteopenia” or “osteopenic”
   if BMD T-score≥−1 then density score=“Normal”
the bone texture status can be determined according to the value of TBS, the values indicated below are examples, as the settings algorithm are adapted to the technical and clinical information:
   if TBS>1.31 then Bone texture status=“Normal”
   if TBS in [1.23, 1.31] then Bone texture status=“Partially Degraded”
   if TBS<1.23 then Bone texture status=“Degraded”
the global score 16 can be determined (as illustrated by FIG. 2) according to the following rules:
   if density score=“Normal” and Bone texture status=“Normal”, then global score=1
   if density score=“Normal” and Bone texture status=“Partially Degraded”, then global score=2
   if density score=“Normal” and Bone texture status=“Degraded”, then global score=3
   if density score=“Osteopenia” or “osteopenic” and Bone texture status=“Normal”, then global score=4
   if density score=“Osteopenia” or “osteopenic” and Bone texture status=“Partially Degraded”, then global score=5
   if density score=“Osteopenia” or “osteopenic” and Bone texture status=“Degraded”, then global score=6
   if density score=“Osteoporosis” or “osteoporotic” and Bone texture status=“Normal”, then global score=7
   if density score=“Osteoporosis” or “osteoporotic” and Bone texture status=“Partially Degraded”, then global score=8
   if density score=“Osteoporosis” or “osteoporotic” and Bone texture status=“Degraded”, then global score=9
training the first artificial intelligence ANN₁ (during the preprocessing phase and the deep learning or training phase) by providing, to the first artificial intelligence the second type of training image 19 (step 7) of the first training set with its associated ground truth 1 comprising or consisting of the global score 16 determined for the training image 9 of the first type of the first training set associated with this training image 19 of the second type of the first training set. In a preferred embodiment of method 100:
   ANN₁ is (but not restricted to) MLP, CNN with several layers (for example 50 layers or more). In a preferred embodiment ANN₁ is a multi-class classifier convolutional neural network;
   This training step is done by gradient backpropagation by Optimizing precision (TP/(TP+FP)) (TP=True Positive, FP=False positive)
   the training is done until minimal test loss (categorical cross-entropy loss function) reached without overfitting.

For the first training set, the first type of training image 9 and the second type of training image 19 are acquired on the same training bone and are acquired less than 6 months apart.

For the first training set, the first type of training image 9 is a dual x-ray absorptiometry (DXA) image, a peripheral quantitative computed tomography ((p)QCT) image and/or High Resolution peripheral quantitative computed tomography (HR-pQCT) image, a computerized tomography (CT) image, or a quantitative ultrasound (QUS) image.

For the first training set, the second type of training image 19 is not a dual x-ray absorptiometry (DXA) image, a peripheral quantitative computed tomography ((p)QCT) image and/or High Resolution peripheral quantitative computed tomography (HR-pQCT) image, a computerized tomography (CT) image, or a quantitative ultrasound (QUS) image, but is for example DICOM (for “Digital imaging and communications in medicine”) image.

For the first training set, the second type of training image 19 is preferably a digital x-ray image, having a spatial resolution per pixel of less than 1 mm.

Method 100 also comprises, before the analysis steps 11, 12 and 13, the following steps:
constructing a second training set by implementing several times the following steps:
   Obtaining a first type 9 of training image (of the second training set) showing a trabecular part of a training bone (during the repository extraction phase)
   Obtaining an associated second type 19 of training image (of the second training set) that is a x-ray based image, showing the same training bone, but not necessary its trabecular part (during the repository extraction phase)
   Determining (during the ground truth processing phase), by technical means (typically comprising or consisting of at least one computer, one central processing or computing unit, one analogue electronic circuit (preferably dedicated), one digital electronic circuit (preferably dedicated) and/or one microprocessor (preferably dedicated) and/or by software means), from the first type of training image 9 of the second training set (but without implementing any artificial intelligence on the image 9):
      a density score 14 depending on or equal to a bone mineral density of the training bone showed on the first type of training image 9 of the second training set; typically, the bone mineral density is determined from the first type of training image directly as a metadata of the DXA the first type of training image; and/or
      a trabecular bone score (TBS) 15 depending on a texture of the trabecular part of the training bone showed on the first type of training image 9 of the second training set; typically, this TBS 15 is determined by calculating or determining an experimental variogram of the gray levels of the first type of training image (patent reference EP1576526_A1)
training the second artificial intelligence ANN₂ (during the preprocessing phase and the deep learning or training phase) by providing, to the second artificial intelligence the second type of training image 19 of the second training set (step 8) with its associated ground truth 2 comprising or consisting of:
   the density score 14 determined for the training image 9 of the first type of the second training set associated with this training image 19 of the second type of the second training set and/or
   the trabecular bone score 15 determined for the training image 9 of the first type of the second training set associated with this training image 19 of the second type of the second training set.

In a preferred embodiment of method 100:
ANN₂ is (but not restricted to) MLP, CNN with several layers (for example 50 layers or more). In a preferred embodiment ANN₁ is a regression convolutional neural network
This training step is done by gradient backpropagation by minimizing quadratic loss functions until minimal test loss reached without overfitting
This training is done until minimal test loss (quadratic loss function) reached without overfitting.

For the second training set, the first type of training image and the second type of training image are acquired on the same training bone and are acquired less than 6 months apart.

For the second training set, the first type of training image is a dual x-ray absorptiometry (DXA) image, a peripheral quantitative computed tomography ((p)QCT) image and/or High Resolution peripheral quantitative computed tomography (HR-pQCT) image, a computerized tomography (CT) image, or a quantitative ultrasound (QUS) image.

For the second training set, the second type of training image is not a dual x-ray absorptiometry (DXA) image, a peripheral quantitative computed tomography ((p)QCT) image and/or High Resolution peripheral quantitative computed tomography (HR-pQCT) image, a computerized tomography (CT) image, or a quantitative ultrasound (QUS) image, but is for example DICOM image.

For the second training set, the second type of training image 19 is preferably a digital x-ray image, having a spatial resolution of less than 1 mm per pixel.

The determined trabecular bone score 15 is a textural parameter which quantifies the local variations in gray level, and is derived from the evaluation of the experimental variogram of the gray levels of the digitized image 9 of the first type. This digitized image 9 is typically a Dual X-ray Absorptiometry (DXA) image.

For these steps for training the first artificial intelligence ANN₁ and for training the second artificial intelligence ANN₂, the trabecular bone score (TBS) 15 is determined from each training image 9 of the first type without implementing any artificial intelligence on the image 9, typically according to the method described in patent application EP1576526_A1, or according to the following steps a) to i) of the training method:
a) determining an optimized pixel sampling S of the image 9 for which each pixel Pᵢ=(xᵢ, yᵢ)ϵS has (or is defined by) its gray level value h(Pᵢ).

From the image 9, a pixel sampling S is determined to select the locations on which the variogram Vᵢ is evaluated (in step d)).

Those locations are optimized so that the final TBS is adapted and efficient in a given clinical context and anatomical site. In the most basic sense, the sampling region corresponds to the region in which the bone is present in the image.

Depending on the bone site, a sub-sample of this region in which the bone is present in the image may be used to increase the performance.
b) for at least one region of interest (ROI) of the pixel sampling S, preferably for a plurality of ROI:
   determining a computation distance R₀ depending on the given region of interest (ROI); The range R₀ is determined depending on the bone skeletal site and the image resolution of image 9. On DXA systems, the value R₀ is between 1 cm and 2 cm.
   choosing a predetermined set of directions I, depending on the given region of interest (ROI). This step of choosing predetermined set of directions I is done by determining a set of N directional unit vectors U={\vec{u_{θ}_{₁}}; . . . , {right arrow over (u_{θ}_{_{N}})}} (N being a positive integer number), where θₖϵ[−π, π], ∀k∈1, . . . , N, θₖ being the angle, around a considered pixel, carrying the vector \vec{u_{θ}_{ₖ}}.

Typically N>2.

The maximum value of N depends on the complexity of the bone structure of the considered imaged bone of the ROI and its image resolution. Typically N<9 for a bone having a non-complex bone structure such like vertebra or lumbar spine, but in some cases N can increase significantly for complex structures such as the proximal femur.

For example: N=3 or 4 or 6 or 8.

The N directional vectors are preferably distributed uniformly at an angle 2ω̅/N around the considered pixel.

The predetermined set of directions I (vectors U={\vec{u_{θ}_{₁}}; . . . , {right arrow over (u_{θ}_{_{N}})}}) depends on:
a skeletal site of a bone on the image and/or on the ROI, the human or animal tissue being the bone, and/or
the considered region of interest (ROI), and/or
a resolution of the image, and/or
a signal/noise ratio of the image.

This determination of a set of directions I:
improves the ability to differentiate or predict a bone fracture, and/or
allows a better measurement reproducibility (or precision), and/or
allows a better correlation with the micro architecture of the bone.

Thus, to compute the variogram V_{P}_{ᵢ} in later step d), the pixel with value h(Pᵢ) is compared to pixels located along lines with specific directions.

Those directions depend both on the type of bone (i.e. skeletal site) and the region of interest selected for measurement on this bone to optimize texture measurements.

Indeed, the selected direction(s) are linked to the morphology of the bone, especially the direction of the trabeculae of the cancellous bone. For example at the spine, the preferred direction of the trabeculae is vertical, so the selected directions will be vertical and horizontal [−π/2, 0, π/2, π] (parallel and perpendicular to the orientation of the trabeculae).
c) for each pixel Pᵢ=(xᵢ, yᵢ)ϵS and each direction \vec{u_{θ}_{ₖ}}ϵU, moving along \vec{u_{θ}_{ₖ}} to a distance rϵ[1, R₀] (in pixels). We note h(Pᵢ+r*\vec{u_{θ}_{ₖ}}) the gray value of such pixel,
d) for each pixel: computing at least one variogram of the gray levels of the sampling S as a function of the distance r along those directions I (i.e. by moving from this pixel by at least one distance rϵ[1, R₀]; as explained previously, moving by a distance r is done by, for each pixel Pᵢ=(xᵢ, yᵢ)ϵS and each direction \vec{u_{θ}_{ₖ}}ϵU, moving along \vec{u_{θ}_{ₖ}} to a distance rϵ[1, R₀] in pixels, h(Pᵢ+r*\vec{u_{θ}_{ₖ}}) being the gray value of such pixel), a variogram being computed:
   for each predetermined direction one by one (i.e. one variogram per pixel in S and per direction among I); if a variogram is computed for each predetermined direction, h(O) being the gray level of an initial given pixel before moving, h(r) being the gray level of a given new pixel after moving by a distance r along one of the predetermined directions from the initial given pixel, the variogram being computed with the formula: Vᵢ(r)=[h(r)−h(O)]² where i□I; or
   for all the predetermined directions at the same time (i.e. one variogram per pixel in S); If a variogram is computed for all the predetermined directions at the same time, the step of computing the variogram of the gray levels as a function of the distance r is done by averaging the squared differences of h over several pairs of pixels, each at distance r with the formula:

$${V}_{{P}_{i}} ( r ) = \frac{1}{N} * {{\sum }_{k = 1}^{k = N} [ h ( {P}_{i} ) - h ( {P}_{i} + r * \vec{{u}_{{θ}_{k}}} ) ]}^{2} ,$$

V_{P}_{ᵢ}(r) being computed for every pixel PᵢϵS.

The formula for V_{P}_{ᵢ} is applied to each PᵢϵS for a given range of values rϵ[1, R₀]. This specific range of values for r is selected to allow V_{P}_{ᵢ}: r→V_{P}_{ᵢ}(r) to converge for the evaluation of all the required parameters of the variogram V_{P}_{ᵢ}.

The range R₀ is determined depending on the bone skeletal site and the image resolution. On DXA systems, the value R₀ is between 1 cm and 2 cm.

The range of computation R₀ is not to be confused with the range parameter c of the variogram model.
e) computing V_{P}_{ᵢ}(r) for every pixel PᵢϵS, and tracing or calculating or determining the associated curves on a log-log scale.

The representation of the variogram curve V_{P}_{ᵢ} in a log-log scale implies that the values along each axis no longer have units.
f) evaluating the full model V as a least squares regression model of V_{P}_{ᵢ} representations,
g) evaluating the parameters of this model including but not limited to the initial slope a, the sill b, the range c, the nugget d, and/or the area under the curve e.

For each variogram V_{P}_{ᵢ} of each pixel and/or for the global variogram V of the sampling S combining the variograms for each pixel, evaluating at least one of the following parameters on a log-log scale:
the initial slope a of the variogram (referenced 54 in FIG. 8),
the sill b, representing the asymptote value of the variogram (referenced 55 in FIG. 8),
the range c, representing the distance at which the variogram curve transitions from a quasi-linear progression to an asymptotic behavior (referenced 56 in FIG. 8),
the nugget d, representing the initial value of the variogram (referenced 57 in FIG. 8), and
the area under the variogram curve e of the variogram (referenced 58 in FIG. 8).

On the log-log plots of V_{P}_{ᵢ} or V, we fit a mathematical model. These parameters (i.e. coefficients) a, b, c, d, e of this model are combined to create the bone texture score B.

Each parameter a, b, c, d, and/or e is evaluated from a least squares regression model of the considered variogram.

Depending on the contents of the image 9, the selected coefficients of the model may vary, because they may not be clearly defined (for example, the variogram curve may not converge to an asymptote, and thus “range” might not be defined)

Preferably, the training method comprises for each variogram of each pixel and/or for a global variogram of the sampling S combining the variograms for each pixel, evaluating at least one of the parameters sill b, range c, the nugget d, area e on a log-log scale.

Preferably, the training method comprises for each variogram of each pixel and/or for a global variogram of the sampling S combining the variograms for each pixel, evaluating at least two of the parameters slope a, sill b, range c, the nugget d, area e on a log-log scale.

Preferably, the training method comprises for each variogram of each pixel and/or for a global variogram of the sampling S combining the variograms for each pixel, evaluating the initial slope a and at least one of the following parameters sill b, range c, the nugget d, area e on a log-log scale.

Preferably, the training method comprises for each variogram of each pixel and/or for a global variogram of the sampling S combining the variograms for each pixel, evaluating all the parameters slope a, sill b, range c, the nugget d, area e on a log-log scale.
h) combining the at least one parameter(s) a, b, c, d and/or e into the TBS (unitless), for example by using linear or nonlinear equations depending on clinical context.

For example, the training method can comprise the step of combining:
the at least one evaluated parameter(s) a, b, c, d and/or e obtained for the variogram of a or each pixel into a TBS for the or each pixel, and/or
the at least one evaluated parameter(s) a, b, c, d and/or e obtained for the global variogram of sampling S into a TBS for the sampling S, and/or
the at least one evaluated parameter(s) a, b, c, d and/or e obtained for the variogram of each pixel into a TBS for the sampling S

At least two parameters among a, b, c, d, e are preferably combined into the TBS using linear or nonlinear equations depending on a clinical context. Indeed, the parameters of the variogram model are combined together into the TBS, using combination equations. As an example, such combination equations could include but not be restricted to a multiple linear model for a given clinical context and anatomical site. In such case scenario, TBS would be defined as:

$$TBS = α a + β b + γ c + δ d + ε e ,$$

where coefficients α, β, γ, δ, ε are respectively associated with the slope, the sill, the nugget and the area under the curve. These coefficients are carefully defined beforehand during clinical performance optimization phases.

These coefficients are typically obtained from different experimental analysis.

Selection of the best coefficients is obtained for example on a large set of clinical studies and images, and further to a grid search optimization phase.

More generally α, β, γ, δ, and/or ε can be obtained in different manners including:
corrective abacus based on experimental measures, and/or
mathematical model and/or simulations of correction based on theory, and/or
machine learning or Artificial Intelligence (AI) algorithms (this machine learning or Artificial Intelligence (AI) being not applied on training image 9), and/or
selection of the best coefficients obtained for example on a large set of clinical studies and images, and further to a grid search optimization phase with clinical performance outcome, and/or
and the combination of the above methods,
etc.

For example:

$$TBS = a$$

$$i . e α = 1 and β = γ = δ = ε = 0$$

Preferably, TBS is calculated from a global variogram of sampling S computed for all the predetermined directions at the same time.
i) optionally, applying robustness improvement step(s) related to the at least one patient factor and/or the at least one technical factor into the TBS, preferably related to both patient and technical factors.

The robustness step(s) may be implemented:
before or during previous step h) (by determining and/or correcting R₀, α, β, γ, δ, ε, a, b, c, d, and/or e, as a function of patient and/or technical factor(s), before or during the determination of calculation of score B), and/or
after previous step h) (by correcting, as a function of patient and/or technical factor(s), score B).

The robustness step(s) may be implemented (by determining and/or correcting R₀, α, β, γ, δ, ε, a, b, c, d, e, and/or B) in different manners including:
corrective abacus based on experimental measures taking into account the patient and/or technical factor(s) and their observed effect on the variogram and/or TBS, and/or
mathematical model and/or simulations of correction based on theory taking into account the patient and/or technical factor(s) and their predicted effect on the variogram and/or TBS, and/or
machine learning or Artificial Intelligence (AI) algorithms learning to minimize the effect on the variogram and/or texture score B of the patient and/or technical factor(s) (this machine learning or Artificial Intelligence (AI) being not applied on training image 9), and/or
selection of the best coefficients obtained for example on a large set of clinical studies and images, and further to a grid search optimization phase with clinical performance outcome, up to the point where the patient and/or technical factor(s) have minimized effect on the variogram and/or TBS, and/or
and the combination of the above methods.

For example, R₀ is determined and/or corrected as a function of the image resolution of the X-Ray acquired image.

The at least one patient factor comprise:
effect of patient morphology including at least one among:
   effect of soft tissue, and/or tissue thickness, and/or its distribution and/or its composition in the patient, and/or indirect surrogates and/or
   a weight and/or Body Mass Index (BMI) and/or belly circumference of the patient, and/or
   a size of the patient, and/or
effect of at least one pathology or condition of the patient (arthrosis, ascites, aortic calcification, gas, etc.), and/or
effect of patient positioning during the acquisition of the image.

The at least one technical factor comprise:
potential defective detectors and sensors for acquiring the image, and/or
effect of scan mode and settings for acquiring the image, and/or
technical characteristics of the imaging device which is used for acquiring the image
effect of the variability in between imaging systems for acquiring the image, and/or
the Signal-Noise-Ratio (SNR) of the image, and/or
the resolution of the image.

The robustness improvement step allows:
to be less influenced by the physiological characteristics of the patient, for example the volume and/or nature of the soft tissues surrounding the bone, and/or
to be less influenced by the choice of the technical parameters of the image acquisition

Typically:
coefficient α:
   Impact the reproducibility
   Optimize fracture prediction
coefficient β:
   Impact the contribution of the global mineralization of a (bone) tissue
coefficient γ:
   Influence the contribution of the overall geometric properties of a (bone) tissue
coefficient δ:
   Is linked to the signal/noise ratio of the image
coefficient ε:
   Impact the reproducibility

The first artificial intelligence and the second artificial intelligence are trained using a same database of first type of training images 9 and second type of training images 19. This qualified dataset is necessary for training the deep learning models of ANN₁ and ANN₂, as they rely on supervised learning.

Such dataset is used for the elaboration, training, and validation of the artificial neural networks (ANN₁ and ANN₂).

Each element of the training dataset is composed of an X-ray digital radiograph associated to a specific ground truth.

The ground truth 1, 2 comes from bone density and bone texture parameters extracted from DXA scans This is also possible with other technologies such as (but not limited to) (p)QCT, CT, QUS images. On those scans the BMD T-scores and bone texture (e.g. TBS) values are retrieved. For a given patient, DXA scans from multiple anatomical sites are used (spine, hip, forearm). The lowest BMD T-score is selected as the most relevant to the fracture risk profile. The BMD T-scores 14 and the bone texture (e.g. TBS) 15 values are compared to their respective classification thresholds (these thresholds or categories are, for BMD “Normal”, “Osteopenia” and “Osteoporosis”, and for TBS “Normal”, “Partially Degraded” and “Degraded” as illustrated in FIG. 2). The resulting stratifications for each score are combined (cf. FIG. 2) to generate a fracture risk category also called global score 16. This fracture risk category is labelled with digits from 1 to 9 (or less if other type of categories is defined).

Depending on the ANN trained, the ground truth 1 or 2 data consists in either fracture risk category labels (ANN₁—multiclass classification task), or continuous values of BMD T-score and Bone texture (e.g. TBS) (ANN₂—regression task).

The matching of the ground truth data with the X-ray digital radiograph is ensured using anonymized Patient IDentifiers (PID). The DXA scans 9 and the X-rays digital radiographs 19 are not necessarily acquired on the same day. However, we ensure that the number of days elapsed between X-ray scan 19 and the DXA scan 9 is sufficiently low (i.e. less than six months) to ensure that the change in bone status between both modalities are minimum.

The ground 1 truth from DXA scans 9 and its associated X-ray digital radiographs 19 do not necessarily originate from the same anatomical site. For simplification purpose, we would use hereunder the term DXA and TBS even though it could be other imaging type of devices and bone texture or structure parameters.

Method 100 is thus based on different approaches. Several artificial neural networks (ANN) models ANN₁ and ANN₂ are defined and trained separately, then combined into one ensemble model to assess the final bone risk category (high risk versus low risk as defined in ground truth of method 100).

One ANN (ANN₁) is designed and trained as a multiclass classifier. It takes as an input a digital X-ray image 6 to predict the risk category class 16 (class 1 to 9 reflecting TBS and BMD DXA measurements 14, 15). The other ANN (ANN₂) is designed to take the same digital X-ray image 6 as input and predicts a set of continuous values of BMD T-score 14 and raw TBS 15.

The usage of both models allows a final bone risk assessment which will be assessed on a second phase with a dedicated ensemble model, based on ANNs predictions (categorical and continuous values) and metadata values 18.

The optimization of these models is ensured with the ultimate goal of minimizing false positive rates and avoiding over-fitting with dedicated training phases. Indeed, models (ANN₁ and ANN₂) have been trained with data-augmentation and cross-validated to get the maximum predictive performance with no bias induced at training.

In this first models' approach of ANN₁, one deep artificial neural network ANN₁ (e.g. including but not limited to Convolutional Neural Network (CNN) or Recurrent Neural Network (RNN)) is implemented and trained to predict “fracture risk profile” classes. On the training phase, ANN₁ is getting as inputs preprocessed digital bone x-ray-based images 19 and their corresponding labels. The dedicated preprocessing of X-rays input images is performed the same way for both training and evaluation. The preprocessing ensures the automation of the ROI selection of the bone while keeping as many resolutions as possible and match the model's input size of method 100. The principle of this deep neural network ANN₁ is to benefit from a strong back-bone architecture and a high-resolution X-ray image to extract the most important feature-maps-information which allow the correct ground-truth risk-profile classification. The output of this classifier consists in a vector of size nine, for which the index of the maximum value is taken as the predicted class.

The second models' approach of ANN₂ is a deep ANN which infers continuous values of min BMD T-score and raw TBS. It can be presented as a deep regression model which outputs a set of two continuous values resulting from a regression output layer. The backbone architecture ensures the input X-ray image is shrunk to a high DXA-like resolution, on which the feature extraction allows the regression and computation task of min BMD T-score and raw TBS.

The preprocessing steps 17 (of the preprocessing phase, testing phase or validation phase) modify or label the X-ray digital images so that they can be fed to the Artificial Neural Networks for inference (for both training and evaluation phases). The preprocessing includes (but not restricted to) the following steps:
labelling of potential artifacts (e.g. metal, implant)
uniformize aspect of the x-ray radiographs (e.g. resolution, CR/BR, size)
Focus/Optimize ROI
Data augmentation (only for the training method)
Identification of the anatomical site
Segmentation
Grayscale pixel-value
Normalization i.e. pixel values in [0, 1]
Quality assessment on radiograph imaging

Method 100 also comprises, before the analysis steps 11, 12 and 13, the following steps (during the testing phase and the clinical optimization phase of FIG. 6):
constructing a third training set by implementing several times the following steps:
   Obtaining a first type of training image 9 (of the third training set) showing a trabecular part of a training bone
   Obtaining a second type of training image 19 (of the third training set) that is a x-ray based image, showing the same training bone but not necessary its trabecular part
   Obtaining metadata 18
   Determining, by technical means, from the first type of training image 9 of the third training set (but without implementing any artificial intelligence on image 9):
      a density score 14 depending on or equal to a bone mineral density of the training bone showed on the first type of training image of the third training set; typically, the bone mineral density is determined from the first type of training image directly as a metadata of the DXA the first type of training image; and
      a trabecular bone score (TBS) 15 depending on a texture of the trabecular part of the training bone showed on the first type of training image 9 of the third training set; typically, this TBS 15 is determined by calculating or determining an experimental variogram of the gray levels of the first type of training image (patent reference EP1576526_A1) (see also steps a) to i) previously described)
   Determining, by technical means (but without implementing any artificial intelligence on image 9), from:
      the density score 14 depending on a bone mineral density of the training bone showed on the first type of training image 9 of the third training set, and
      the trabecular bone score (TBS) 15 depending on a texture of the trabecular part of the training bone showed on the first type of training image 9 of the third training set
   a global score 16 depending on this density score and trabecular bone score
   implementing the first and second analysis 11, 12 by the first and second artificial intelligence on the second type of training image of the third training set, and
training the third artificial intelligence by learning from:
   a difference between the scores (density score 14, TBS 15 and/or global score 16) obtained from the first type of training image 9 of the third training set without ANN₁ and ANN₂ and the scores (density score 14, TBS 15 and/or global score 16) obtained from the second type of training image 19 of the same training bone of the third training set with ANN₁ and ANN₂, and
   Metadata 18
      In a preferred embodiment of method 100:
AI₃ is a classification And Regression Tree (CART) to assess if flag or no flag with associated confidence score
This training step is done by training with supervised learning from DXA ground truth (flag or no flag)
this training is done using grid search on tree architecture to optimize precision score (optimize vertical depth, number of terminal nodes, max features to consider for splitting nodes, etc.) until minimal test loss reached without overfitting.

The first training set and the second training set can be the same training set.

The third training set is not the same training set than the first training set and/or than the second training set, because the third training set (used during clinical optimization phase) is used to optimize method 100 after the training of ANN₁ and ANN₂ based on the first training set and/or the second training set.

The first, second and third artificial intelligences are trained separately.

The validation phase (left part of FIG. 7) is important to prepare and validate all the ANN; (i=1 or 2) models which have been fully optimized during the phase described above and relative to the clinical aspects. In this phase, the models of ANN₁ and ANN₂ are tested on external cohorts to confirm their robustness and their ability to predict the final clinical outcome.

During this validation phase all the models, their configurations, their parameters, are sealed, timestamped, traced and packaged. The mechanism used to ensure the traceability and the tracking of the parameters, is blockchain like. It is logging all the parameters, the models, the metadata, the code versions, the metrics, the tags, the labels, the output files, and the results into timestamped records. The steps involved in the validation phase is ensuring the persistence of all the configurations of the ANNi models as records.

All these operation steps of this phase meet the regulatory requirements and their compliance in the field of software as medical device which is embedding AI and ANN technologies. It is also following the best technology practices in this domain.

At the end of this phase, all the ANNi models are successfully validated and ready for their integration into the final product during the industrialization phase.

Once all the ANNi models have been validated during the validation phase, the final global module is integrated inside the final product for industrialization phase (FIG. 1 and right part of FIG. 7).

The traceability of all the ANNi training experiments is ensured during the previous phases, from the ground truth until deployment. This traceability principle is warrantying the adequation of the models with their optimized parameters for clinical validation into the final product. It is also allowing the adequate deployed module version into the field with the possibility to update or upgrade this adequate module when better optimizations are performed during the off-line continuous improvement flow.

This module is loaded dynamically into the product providing the new features as a service as per this description of this invention.

The device according to the invention comprise technical means (in particular means arranged for and/or programmed to and/or configured to respectively calculate, determine, obtain, choose, compute, evaluate, combine, apply improvement step(s), receive an image, implement an artificial intelligence, implement an analysis, give a result, construct a training set, train an artificial intelligence) arranged and/or programmed to and/or configured to implement all the previously described steps (in particular the steps of respectively calculating, determining, obtaining, choosing, computing, evaluating, combining, applying improvement step(s), receiving an image, implementing an artificial intelligence, implementing an analysis, giving a result, constructing a training set, training an artificial intelligence)

Typically, at least one of the means of the device according to the invention previously described, preferably each of the means of the device according to the invention (and in particular the means arranged for and/or programmed to and/or configured to calculate, determine, obtain, choose, compute, evaluate, combine, apply improvement step(s), receive an image, implement an artificial intelligence, implement an analysis, give a result, construct a training set, train an artificial intelligence), are technical means.

Typically, each of the means of the device according to the invention implementing the steps previously described (and in particular the means arranged for and/or programmed to and/or configured to calculate, determine, obtain, choose, compute, evaluate, combine, apply improvement step(s), receive an image, implement an artificial intelligence, implement an analysis, give a result, construct a training set, train an artificial intelligence) comprise at least one computer, one central processing or computing unit, one analogue electronic circuit (preferably dedicated), one digital electronic circuit (preferably dedicated) and/or one microprocessor (preferably dedicated) and/or software means.

The device according to the invention comprises:
Means for implementing the step(s) of acquiring the image 6 and/or 9 and/or 19: these means for acquiring the digitized image typically comprise:
   conventional x-ray imaging system, and/or
   digital x-ray imaging system, and/or
   Dual X-ray Absorptiometry (DXA) imaging system, and/or
   projected Computed Tomography (CT) imaging system, and/or
   Quantitative computed tomography (QCT) imaging system, and/or
   projected Quantitative computed tomography imaging system, and/or
   peripheral Quantitative computed tomography (pQCT) imaging system, and/or
   High-Resolution peripheral Quantitative computed tomography (HR-pQCT) imaging system, and/or
   a combination thereof, and
means for implementing the previously described steps (in particular the steps of respectively calculating, determining, obtaining, choosing, computing, evaluating, combining, applying improvement step(s), receiving an image, implementing an artificial intelligence, implementing an analysis, giving a result, constructing a training set, training an artificial intelligence): these means are typically grouped together in a single computer
a screen (arranged for displaying the output 16 of ANN₁ and/or the outputs 14 and/or 15 of ANN₂ and/or the conclusion or output of AI₃.

This embodiment also comprises:
a computer program comprising instructions which, when executed by a computer, implement the steps of the method 100, and/or
a computer program product comprising instructions which, when the program is executed by a computer, cause the computer to carry out the steps of the method 100, and/or
a computer-readable storage medium comprising instructions which, when executed by a computer, cause the computer to carry out the steps of the method 100.

Of course, the invention is not limited to the examples which have just been described and numerous amendments can be made to these examples without exceeding the scope of the invention.

For example, a variant of the method 100 (and the corresponding device) can comprise only ANN₁ (without ANN₂ and AI₃) or only ANN₂ (without ANN₁ and AI₃). In this variant, method 100 (described only for tits differences compared to the previous description of FIGS. 1 à 8) is a method for analyzing a texture of a bone from the digitized image 6, obtained by imaging and chosen in a region comprising a bone structure, comprising:
receiving the input x-ray image 6 showing an input bone,
a bone score analysis (respectively 11 or 12 previously described) of the received input x-ray image 6 by a bone score artificial intelligence (respectively ANN₁ or ANN₂) implemented by technical means, the bone score artificial intelligence giving as a result of this bone score analysis:
   the global score 16 depending at least on a trabecular bone score (TBS) depending on a texture of the trabecular part of the input bone showed on the received input x-ray image (i.e. if the bone score artificial intelligence is ANN₁), and/or
   the density score 14 depending on (or consisting of) a bone mineral density of the input bone showed on the received input x-ray image, and/or the trabecular bone score (TBS) 15 depending on a texture of the trabecular part of the input bone showed on the received input x-ray image (i.e. if the bone score artificial intelligence is ANN₂).

The bone score artificial intelligence ANN₁ or ANN₂ is a neural network.

Still in this variant, if the bone score artificial intelligence is ANN₁, method 100 thus comprises, before analysis step 11 the training already described for ANN₁:
constructing the first training set by implementing several times the following steps:
   Obtaining the first type of training image 9 showing a trabecular part of a training bone
   Obtaining the associated second type of training image 12 that is a x-ray based image, showing the same training bone but not necessary its trabecular part
   Determining, by technical means (but without implementing any artificial intelligence on image 9), from the first type of training image 9:
      the density score 14 depending on a bone mineral density of the training bone showed on the first type of training image, and
      the trabecular bone score (TBS) 15 depending on a texture of the trabecular part of the training bone showed on the first type of training image
   Determining, by technical means (but without implementing any artificial intelligence on image 9 and/or on previously determined density score 14 and/or on previously determined TBS 15), from:
      the density score 14 depending on a bone mineral density of the training bone showed on the first type of training image, and
      the trabecular bone score (TBS) 15 depending on a texture of the trabecular part of the training bone showed on the first type of training image
   the global score 16 depending on these density score and trabecular bone score
training the bone score artificial intelligence ANN₁ by providing to the bone score artificial intelligence the second type of training image 19 with its associated ground truth comprising or consisting of the global score 16 determined for the training image 9 of the first type associated with this training image 19 of the second type.

Still in this variant, if the bone score artificial intelligence is ANN₂, method 100 thus comprises, before analysis step 12 the training already described for ANN₂:
constructing a second training set by implementing several times the following steps:
   Obtaining the first type of training image 9 showing a trabecular part of a training bone
   Obtaining the associated second type of training image 19 that is a x-ray based image, showing the same training bone but not necessary its trabecular part
   Determining, by technical means (but without implementing any artificial intelligence on image 9), from the first type of training image:
      the density score 14 depending on a bone mineral density of the training bone showed on the first type of training image, and/or
      the trabecular bone score (TBS) 15 depending on a texture of the trabecular part of the training bone showed on the first type of training image
training the bone score artificial intelligence by providing to the bone score artificial intelligence the second type of training image with its associated ground truth comprising or consisting of the density score determined for the training image 9 of the first type associated with this training image 19 of the second type and/or the trabecular bone score determined for the training image 9 of the first type associated with this training image 19 of the second type.

In another variant, method 100 (and the corresponding device) can comprise ANN₁ and ANN₂ without AI₃.

Of course, the different characteristics, forms, variants and embodiments of the invention can be combined with each other in various combinations to the extent that they are not incompatible or mutually exclusive.

1-40. (canceled)

41. A method for analyzing a texture of a bone, comprising:
receiving a digitized input x-ray image showing an input bone,
a bone score analysis of the received input x-ray image by a bone score artificial intelligence implemented by technical means, the bone score artificial intelligence giving as a result of this bone score analysis, but without any calculation or determination of an experimental variogram of the gray levels of the received input x-ray image:
a global score depending at least on a trabecular bone score which quantifies the local variations in gray levels from the experimental variogram of the gray levels of the trabecular part of the input bone showed on the received input x-ray image, and/or
a trabecular bone score which quantifies the local variations in gray levels from the experimental variogram of the gray levels of the trabecular part of the input bone showed on the received input x-ray image.

42. The method according to claim 41, wherein the bone score artificial intelligence is a neural network.

43. The method according to claim 41, comprising:
constructing a training set by implementing several times the following steps:
obtaining a first type of training image showing a trabecular part of a training bone,
obtaining an associated second type of training image that is an x-ray based image, showing the same training bone,
determining, by technical means, from the first type of training image:
a density score depending on a bone mineral density of the training bone showed on the first type of training image, and
a trabecular bone score depending on a texture of the trabecular part of the training bone showed on the first type of training image,
determining, by technical means, from:
the density score depending on a bone mineral density of the training bone showed on the first type of training image, and
the trabecular bone score depending on a texture of the trabecular part of the training bone showed on the first type of training image,
a global score depending on these density score and trabecular bone score, and
training the bone score artificial intelligence by providing to the bone score artificial intelligence the second type of training image with its associated ground truth comprising the global score determined for the training image of the first type associated with this training image of the second type.

44. The method according to claim 41, comprising:
constructing a training set by implementing several times the following steps:
obtaining a first type of training image showing a trabecular part of a training bone,
obtaining an associated second type of training image that is an x-ray-based image, showing the same training bone,
determining, by technical means, from the first type of training image:
a density score depending on a bone mineral density of the training bone showed on the first type of training image, and/or
a trabecular bone score depending on a texture of the trabecular part of the training bone showed on the first type of training image,
training the bone score artificial intelligence by providing to the bone score artificial intelligence the second type of training image with its associated ground truth comprising:
the density score determined for the training image of the first type associated with this training image of the second type and/or
the trabecular bone score determined for the training image of the first type associated with this training image of the second type.

45. The method according to claim 41, comprising:
receiving the input x-ray image showing an input bone,
a first analysis of the received input x-ray image by a first artificial intelligence implemented by technical means, the first artificial intelligence giving as a result of the first analysis a global score depending both:
on a density score depending on a bone mineral density of the input bone showed on the received input x-ray image, and
on a trabecular bone score depending on a texture of the trabecular part of the input bone showed on the received input x-ray image,
a second analysis of the received input x-ray image by a second artificial intelligence implemented by technical means, the second artificial intelligence giving as a result of the second analysis:
the density score depending on a bone mineral density of the input bone showed on the received input x-ray image, and/or
the trabecular bone score depending on a texture of the trabecular part of the input bone showed on the received input x-ray image,
a third analysis, by a third artificial intelligence implemented by technical means, the third artificial intelligence having as input the results of the first and second analysis and having as output a result depending on the consistency between the result of the first analysis and the result of the second analysis.

46. The method according to claim 45, wherein the third artificial intelligence uses as further input at least one parameter among: age of the patient on whom the received input x-ray image was acquired, gender of the patient on whom the received input x-ray image was acquired, morphotype of the patient on whom the received input x-ray image was acquired, machine type with which the received input x-ray image was acquired, and/or acquisition parameter(s) of the machine with which the received input x-ray image was acquired.

47. The method according to claim 45, wherein the first artificial intelligence is a neural network and the second artificial intelligence is a neural network.

48. The method according to claim 45, wherein the first artificial intelligence and the second artificial intelligence and the third artificial intelligence are three distinct artificial intelligences.

49. The method according to claim 45, wherein the technical means for implementing the first and second and third artificial intelligences are the same technical means.

50. The method according to claim 45, comprising:
constructing a first training set by implementing several times the following steps:
obtaining a first type of training image showing a trabecular part of a training bone,
obtaining an associated second type of training image that is a x-ray based image, showing the same training bone,
determining, by technical means, from the first type of training image:
a density score depending on a bone mineral density of the training bone showed on the first type of training image, and
a trabecular bone score depending on a texture of the trabecular part of the training bone showed on the first type of training image
determining, by technical means, from:
the density score depending on a bone mineral density of the training bone showed on the first type of training image, and
the trabecular bone score depending on a texture of the trabecular part of the training bone showed on the first type of training image,
a global score depending on these density score and trabecular bone score, and
training the first artificial intelligence by providing, to the first artificial intelligence the second type of training image with its associated ground truth comprising the global score determined for the training image of the first type associated with this training image of the second type.

51. The method according to claim 45, comprising:
constructing a second training set by implementing several times the following steps:
obtaining a first type of training image showing a trabecular part of a training bone,
Obtaining an associated second type of training image that is a x-ray based image, showing the same training bone, and
determining, by technical means, from the first type of training image:
a density score depending on a bone mineral density of the training bone showed on the first type of training image, and/or
a trabecular bone score depending on a texture of the trabecular part of the training bone showed on the first type of training image,
training the second artificial intelligence by providing, to the second artificial intelligence the second type of training image with its associated ground truth comprising:
the density score determined for the training image of the first type associated with this training image of the second type and/or
the trabecular bone score determined for the training image of the first type associated with this training image of the second type.

52. The method according to claim 50, wherein the first artificial intelligence and the second artificial intelligence are trained using a same database of first type of training images and second type of training images.

53. The method according to claim 45, comprising
constructing a third training set by implementing several times the following steps:
obtaining a first type of training image showing a trabecular part of a training bone,
obtaining a second type of training image that is a x-ray based image, showing the same training bone,
determining, by technical means, from the first type of training image:
a density score depending on a bone mineral density of the training bone showed on the first type of training image, and
a trabecular bone score depending on a texture of the trabecular part of the training bone showed on the first type of training image,
determining, by technical means, from:
the density score depending on a bone mineral density of the training bone showed on the first type of training image, and
the trabecular bone score depending on a texture of the trabecular part of the training bone showed on the first type of training image,
a global score depending on this density score and trabecular bone score, and
implementing the first and second analysis by the first and second artificial intelligence on the second type of training image, and
training the third artificial intelligence by learning from a difference between the scores obtained from the first type of training image and the scores obtained from the second type of training image of the same training bone.

54. The method according to claim 50, wherein the first, second and third artificial intelligences are trained separately.

55. The method according to claim 43, wherein the first type of training image and the second type of training image are acquired on the same training bone and are acquired less than 6 months apart.

56. The method according to claim 43, wherein the first type of training image is a dual x-ray absorptiometry image, a peripheral quantitative computed tomography image and/or High Resolution peripheral quantitative computed tomography image, a computerized tomography image, or a quantitative ultrasound image.

57. The method according to claim 43, wherein the second type of training image is not a dual x-ray absorptiometry image, a peripheral quantitative computed tomography image and/or High Resolution peripheral quantitative computed tomography image, a computerized tomography image, or a quantitative ultrasound image.

58. The method according to claim 41, wherein the received input x-ray image is not a dual x-ray absorptiometry image, a peripheral quantitative computed tomography image and/or High Resolution peripheral quantitative computed tomography image, a computerized tomography image, or a quantitative ultrasound image.

59. The method according to claim 41, wherein the received input x-ray image is a digital x-ray image, having a spatial resolution of less than 1 mm per pixel.

60. A device for analyzing a texture of a bone, comprising:
means arranged to and/or programmed to and/or configured to receive a digitized input x-ray image showing an input bone,
a bone score artificial intelligence arranged to and/or programmed to and/or configured to implement a bone score analysis of the received input x-ray image, the bone score artificial intelligence being arranged to and/or programmed to and/or configured to give as a result of this bone score analysis, but without any calculation or determination of an experimental variogram of the gray levels of the received input x-ray image:
a global score depending at least on a trabecular bone score which quantifies the local variations in gray levels from the experimental variogram of the gray levels of the trabecular part of the input bone showed on the received input x-ray image, and/or
a trabecular bone score which quantifies the local variations in gray levels from the experimental variogram of the gray levels of the trabecular part of the input bone showed on the received input x-ray image.

61. The device according to claim 60, wherein the bone score artificial intelligence is a neural network.

62. The device according to claim 60, comprising:
means arranged to and/or programmed to and/or configured to construct a training set by implementing several times the following steps:
obtaining a first type of training image showing a trabecular part of a training bone
obtaining an associated second type of training image that is a x-ray based image, showing the same training bone
determining, by technical means, from the first type of training image:
a density score depending on a bone mineral density of the training bone showed on the first type of training image, and
a trabecular bone score depending on a texture of the trabecular part of the training bone showed on the first type of training image
determining, by technical means, from:
the density score depending on a bone mineral density of the training bone showed on the first type of training image, and
the trabecular bone score depending on a texture of the trabecular part of the training bone showed on the first type of training image,
a global score depending on these density score and trabecular bone score, and
means arranged to and/or programmed to and/or configured to train the bone score artificial intelligence by providing to the bone score artificial intelligence the second type of training image with its associated ground truth comprising the global score determined for the training image of the first type associated with this training image of the second type.

63. The device according to claim 60, comprising:
means arranged to and/or programmed to and/or configured to construct a training set by implementing several times the following steps:
obtaining a first type of training image showing a trabecular part of a training bone
obtaining an associated second type of training image that is an x-ray-based image, showing the same training bone
determining, by technical means, from the first type of training image:
a density score depending on a bone mineral density of the training bone showed on the first type of training image, and/or
a trabecular bone score depending on a texture of the trabecular part of the training bone showed on the first type of training image,
means arranged to and/or programmed to and/or configured to train the bone score artificial intelligence by providing to the artificial intelligence the second type of training image with its associated ground truth comprising:
the density score determined for the training image of the first type associated with this training image of the second type and/or
the trabecular bone score determined for the training image of the first type associated with this training image of the second type.

64. The device according to claim 60, comprising:
means arranged to and/or programmed to and/or configured to receive the input x-ray image showing an input bone,
a first artificial intelligence arranged to and/or programmed to and/or configured to implement a first analysis of the received input x-ray image, the first artificial intelligence being arranged to and/or programmed to and/or configured to give as a result of the first analysis a global score depending both:
on a density score depending on a bone mineral density of the input bone showed on the received input x-ray image, and
on a trabecular bone score depending on a texture of the trabecular part of the input bone showed on the received input x-ray image,
a second artificial intelligence arranged to and/or programmed to and/or configured to implement a second analysis of the received input x-ray image, the second artificial intelligence being arranged to and/or programmed to and/or configured to give as a result of the second analysis:
the density score depending on a bone mineral density of the input bone showed on the received input x-ray image, and/or
the trabecular bone score depending on a texture of the trabecular part of the input bone showed on the received input x-ray image, and
a third artificial intelligence arranged to and/or programmed to and/or configured to implement a third analysis, the third artificial intelligence being arranged to and/or programmed to and/or configured to have as input the results of the first and second analysis and to have as output a result depending on the consistency between the result of the first analysis and the result of the second analysis.

65. The device according to claim 64, wherein the third artificial intelligence is arranged to and/or programmed to and/or configured to use as further input at least one parameter among: age of the patient on whom the received input x-ray image was acquired, gender of the patient on whom the received input x-ray image was acquired, morphotype of the patient on whom the received input x-ray image was acquired, machine type with which the received input x-ray image was acquired, and/or acquisition parameter(s) of the machine with which the received input x-ray image was acquired.

66. The device according to claim 64, wherein the first artificial intelligence is a neural network and the second artificial intelligence is a neural network.

67. The device according to claim 64, wherein the first artificial intelligence and the second artificial intelligence and the third artificial intelligence are three distinct artificial intelligences.

68. The device according to claim 64, wherein the technical means for implementing the first and second and third artificial intelligences are the same technical means

69. The device according to claim 64, comprising:
means arranged to and/or programmed to and/or configured to construct a first training set by implementing several times the following steps:
obtaining a first type of training image showing a trabecular part of a training bone,
obtaining an associated second type of training image that is an x-ray based image, showing the same training bone, and
determining, by technical means, from the first type of training image:
a density score depending on a bone mineral density of the training bone showed on the first type of training image, and
a trabecular bone score depending on a texture of the trabecular part of the training bone showed on the first type of training image, and
determining, by technical means, from:
the density score depending on a bone mineral density of the training bone showed on the first type of training image, and
the trabecular bone score depending on a texture of the trabecular part of the training bone showed on the first type of training image
a global score depending on these density score and trabecular bone score, and
means arranged to and/or programmed to and/or configured to train the first artificial intelligence by providing, to the first artificial intelligence the second type of training image with its associated ground truth comprising the global score determined for the training image (9) of the first type associated with this training image (19) of the second type.

70. The device according to claim 64, comprising:
means arranged to and/or programmed to and/or configured to construct a second training set by implementing several times the following steps:
obtaining a first type of training image showing a trabecular part of a training bone
obtaining an associated second type of training image that is an x-ray-based image, showing the same training bone
determining, by technical means, from the first type of training image:
a density score depending on a bone mineral density of the training bone showed on the first type of training image, and/or
a trabecular bone score depending on a texture of the trabecular part of the training bone showed on the first type of training image,
and
means arranged to and/or programmed to and/or configured to train the second artificial intelligence by providing, to the second artificial intelligence the second type of training image with its associated ground truth comprising:
the density score determined for the training image of the first type associated with this training image of the second type and/or
the trabecular bone score determined for the training image of the first type associated with this training image of the second type.

71. The device according to claim 69, wherein the first artificial intelligence and the second artificial intelligence are arranged to and/or programmed to and/or configured to be trained by using a same database of first type of training images and second type of training images.

72. The device according to claim 69, comprising:
means arranged to and/or programmed to and/or configured to construct a third training set by implementing several times the following steps:
obtaining a first type of training image showing a trabecular part of a training bone
obtaining a second type of training image that is an x-ray based image, showing the same training bone
determining, by technical means, from the first type of training image:
a density score depending on a bone mineral density of the training bone showed on the first type of training image, and
a trabecular bone score depending on a texture of the trabecular part of the training bone showed on the first type of training image, and
determining, by technical means, from:
the density score depending on a bone mineral density of the training bone showed on the first type of training image, and
the trabecular bone score (TBS) depending on a texture of the trabecular part of the training bone showed on the first type of training image,
a global score depending on this density score and trabecular bone score,
implementing the first and second analysis by the first and second artificial intelligence on the second type of training image, and
means arranged to and/or programmed to and/or configured to train the third artificial intelligence by learning from a difference between the scores obtained from the first type of training image and the scores obtained from the second type of training image of the same training bone.

73. The device according to claim 69, wherein the first, second and third artificial intelligences are arranged to and/or programmed to and/or configured to be trained separately.

74. The device according to claim 62, wherein the means arranged to and/or programmed to and/or configured to train the first artificial intelligence and the means arranged to and/or programmed to and/or configured to train the second artificial intelligence are arranged together to and/or programmed to and/or configured together to check that the first type of training image and the second type of training image have been acquired on the same training bone and have been acquired less than 6 months apart.

75. The device according to claim 62, wherein the first type of training image is a dual x-ray absorptiometry image, a peripheral quantitative computed tomography image and/or High Resolution peripheral quantitative computed tomography image, a computerized tomography image, or a quantitative ultrasound image.

76. The device according to claim 62, wherein the second type of training image is not a dual x-ray absorptiometry image, a peripheral quantitative computed tomography image and/or High Resolution peripheral quantitative computed tomography image, a computerized tomography image, or a quantitative ultrasound image.

77. The device according to claim 60, wherein the received input x-ray image is not a dual x-ray absorptiometry image, a peripheral quantitative computed tomography image and/or High Resolution peripheral quantitative computed tomography image, a computerized image, or a quantitative ultrasound image.

78. The device according to claim 60, wherein the received input x-ray image is a digital x-ray image, having a spatial resolution of less than 1 mm per pixel.

79. A computer program comprising instructions which, when executed in a computer, implement the steps of the method according to claim 41.

80. A computer-readable storage medium comprising instructions which, when executed by a computer, cause the computer to carry out the steps of the method according to claim 41.
